(19) United States
(12) Patent Application Publication
(10) Pub. No.: US 2014/0329309 A1
(43) Pub. Date: Nov. 6, 2014

(54) Ribosomal Promoters for Production in Microorganisms

(71) Applicant: Danisco US Inc., Palo Alto, CA (US)

(72) Inventors: Cristina Bongiorni, Fremont, CA (US); Bryan P. Fox, Pacifica, CA (US); Anita van Kimmenade, San Bruno, CA (US)

(21) Appl. No.: 14/364,016

(22) PCT Filed: Dec. 6, 2012
(86) PCT No.: PCT/US2012/068285

§ 371 Date: Jun. 9, 2014

Related U.S. Application Data
(60) Provisional application No. 61/569,202, filed on Dec. 9, 2011.
(60) Provisional application No. 61/577,491, filed on Dec. 19, 2011.

(51) Int. Cl. C12N 9/54

(52) U.S. Cl. CPC C12N 9/54; C12Y 304/21062
USPC 435/320.1

(57) ABSTRACT

The present invention provides methods and compositions of improved expression systems in microorganisms. The methods and compositions comprise a ribosomal promoter derived from a Bacillus species microorganism, such a ribosomal RNA or a ribosomal protein promoter.

### PRIORITY

The present application claims priority to U.S. Provisional Application Ser. No. 61/569,202, filed on Dec. 9, 2011 and U.S. Provisional Application Ser. No. 61/577,491, filed Dec. 19, 2011, both of which are hereby incorporated by reference in their entirety.

### FIELD OF THE INVENTION

The present invention relates to the production of proteins in microorganisms. In particular, the present invention provides methods and compositions of improved expression systems in microorganisms. In certain embodiments, the methods and compositions comprise a ribosomal promoter derived from a Bacillus species microorganism.

### BACKGROUND OF THE INVENTION

Genetic engineering has allowed the improvement of microorganisms used as industrial bioreactors or cell factories. For example, Bacillus species produce and secrete a large number of useful proteins and metabolites. The most common Bacillus species used in industry are B. licheniformis, B. amyloliquefaciens, and B. subtilis. Because of their GRAS (generally recognized as safe) status, strains of these Bacillus species are natural candidates for the production of proteins utilized in the food and pharmaceutical industries. Important production enzymes include α-amylases, neutral proteases, and alkaline (or serine) proteases. However, in spite of advances in the understanding of production of proteins in Bacillus host cells, there remains a need for methods to improve the expression and production of these proteins by microorganisms.

Recombinant production of a product encoded by a gene is accomplished by constructing expression vectors suitable for use in a host cell in which the nucleic acid coding for a desired product is placed under the expression control of a promoter. The expression vector is introduced into a host cell by various techniques, such as transformation, and production of the desired product is then achieved by culturing the transformed host cell under suitable conditions necessary for the functioning of the promoter included in the expression vector. While numerous promoters are known in the art, there is a need for new promoters, which improve the expression of heterologous genes and coding sequences.

### SUMMARY OF THE INVENTION

The present invention provides novel promoters, expression vectors, microorganisms, and methods for the production of a nucleic acid coding for a protein of interest. In particular, the present invention provides novel promoters, expression vectors, microorganisms, and methods for the production of a nucleic acid coding for a protein of interest comprising a ribosomal promoter derived from Bacillus subtilis. Ribosomal promoters include, for example, ribosomal RNA promoters and ribosomal protein promoters.

In one embodiment, the invention provides a nucleic acid comprising a B. subtilis ribosomal promoter operably linked to a nucleic acid encoding a protein of interest. In a particular embodiment, the invention provides a nucleic acid comprising a B. subtilis ribosomal RNA promoter operably linked to a nucleic acid encoding a protein of interest. In another embodiment, the invention provides a nucleic acid comprising a B. subtilis ribosomal protein promoter operably linked to a nucleic acid encoding a protein of interest.

In another embodiment, the invention provides an expression vector comprising a nucleic acid comprising a B. subtilis ribosomal promoter operably linked to a nucleic acid encoding a protein of interest. In one embodiment, the expression vector comprises a nucleic acid comprising a B. subtilis ribosomal RNA promoter operably linked to a nucleic acid encoding a protein of interest. In another embodiment, the expression vector comprises a nucleic acid comprising a B. subtilis ribosomal protein promoter operably linked to a nucleic acid encoding a protein of interest.

In another embodiment, the invention provides a microorganism comprising a nucleic acid comprising a B. subtilis ribosomal promoter. In one embodiment, the invention provides a gram positive microorganism comprising a nucleic acid comprising a B. subtilis ribosomal promoter. In one embodiment the ribosomal promoter is a ribosomal RNA promoter. In another embodiment, the ribosomal promoter is a ribosomal protein promoter.

In another embodiment, the invention provides a method for producing a protein of interest comprising culturing a microorganism that comprises a nucleic acid comprising a B. subtilis ribosomal promoter under conditions suitable for the microorganism to produce the protein. In one embodiment the ribosomal promoter is a ribosomal RNA promoter. In another embodiment, the ribosomal promoter is a ribosomal protein promoter.

In another embodiment, the invention provides a method for producing a protein of interest without amplification of an expression construct. In certain embodiments, the method comprises transforming a microorganism with a nucleic acid or vector comprising a ribosomal promoter, wherein the nucleic acid or vector integrates into the host cell as a single integrant, and culturing the microorganism under conditions suitable for the microorganism to produce the protein. In one embodiment the ribosomal promoter is a ribosomal RNA promoter. In another embodiment, the ribosomal promoter is a ribosomal protein promoter.

In certain embodiments, the invention provides a method of producing a protein of interest by introducing a nucleic acid or vector described herein into a host cell so that it integrates into the host cell but does not require the use of an antibiotic marker.

In certain embodiments described herein, the ribosomal RNA promoter is a rrn promoter derived from B. subtilis. In some embodiments, the rrn promoter is a rrnB, rrnI, or rrnE ribosomal RNA promoter from B. subtilis. In a specific embodiment, the ribosomal RNA promoter is a P2 rrnI ribosomal RNA promoter from B. subtilis.

In other embodiments, the ribosomal RNA promoter comprises the nucleotide sequence of any one of SEQ ID NOs: 1-6, a subsequence of any one of SEQ ID NOs: 1-6 that retains promoter activity, a nucleic acid that is at least 60% homologous to any one of SEQ ID NOs: 1-6, or a nucleic acid that hybridizes under medium stringency conditions with any one of SEQ ID NOs: 1-6 or the subsequence thereof that retains promoter activity. In a specific embodiment, the ribosomal RNA promoter comprises the nucleotide sequence of SEQ ID NO: 3 or a subsequence thereof retaining promoter activity. In other embodiments, combinations of any of the above promoters can be used. For example, one or more of a P1, P2, or P3 promoter of a rrnI, rrnB, and rrnE promoters can be used together.

In other embodiments described herein, the ribosomal protein promoter is derived from B. subtilis. In some embodiments, the ribosomal protein promoter is a rpsD or rpsJ ribosomal protein promoter from B. subtilis.

In another embodiment, the ribosomal protein promoter comprises the nucleotide sequence of any one of SEQ ID NOs: 13-14, a subsequence of any one of SEQ ID NOs: 13-14 that retains promoter activity, a nucleic acid that is at least 60% homologous to any one of SEQ ID NOs: 13-14, or a nucleic acid that hybridizes under medium stringency conditions with any one of SEQ ID NOs: 13-14 or the subsequence thereof that retains promoter activity. In other embodiments, combinations of any of the above promoters can be used. For example, one or more promoters of a rpsD or rpsJ promoter can be used together. In other embodiments, the ribosomal protein promoter comprises a nucleic acid that is at least 70%, 80%, 90%, 93%, 95%, 97%, or 99% homologous to any one of SEQ ID NOs: 13-14, or a subsequence thereof that retains promoter activity.

The ribosomal promoters described herein can be operably linked to a nucleic acid encoding a protein of interest. In one embodiment, the protein of interest is selected from the group consisting of a hormone, enzyme, growth factor, reporter gene (e.g., green fluorescent protein), and cytokine. In another embodiment, the protein of interest is an enzyme. An enzyme used in the invention can be, for example, a protease, cellulase, amylase, xylanase, phytase, mannanase, hemicellulase, carbohydrase, hydrolase, esterase, oxidase, permease, pullulanase, laccase, lipase, reductase, isomerase, epimerase, tautomerase, transferase, kinase, and phosphatase. In a particular embodiment, the protein of interest is a protease. In another particular embodiment the protein of interest is a subtilisin. In a specific embodiment, the protein of interest is encoded by SEQ ID NOs: 9, 11, 18 or 20.

A protein of interest can be heterologous or homologous to the microorganism in which it is expressed. In certain embodiments, the nucleic acid, vector, or expression construct that is used to express the nucleic acid encoding the protein of interest is integrated into the host cell. In certain embodiments, the nucleic acid, vector, or expression construct that is used to express the nucleic acid encoding the protein of interest is not integrated into the host cell. The nucleic acid, vector, or expression construct that is used to express the nucleic acid encoding the protein of interest can be amplified in the host cell or it can be maintained as a single copy.

Any bacterial or fungal microorganism that is capable of expression from a ribosomal promoter can be used herein as a host cell. In certain embodiments, the microorganism is a gram positive microorganism. In some embodiments, the microorganism is a member of the genus Bacillus. Examples of Bacillus cells that are useful in the invention include, for example, B. subtilis, B. licheniformis, B. lentus, B. brevis, B. stearothermophilus, B. alkalophilus, B. amyloliquefaciens, B. coagulans, B. circulans, B. lautus, and B. thuringiensis. In other embodiments, the microorganism is E. coli, Pseudomonas spp. (e.g., P. aeruginoa and P. alcaligenes), or Streptomyces spp., (e.g., Streptomyces lividans).

### BRIEF DESCRIPTION OF THE DRAWINGS

FIG. 1 shows organization of the Bacillus subtilis rrn operons used in this study. The different strains constructed from the fusion of the promoters to the target genes are listed in Table 1-2.

FIG. 2 shows the alignment of rrnE P2 with the P1 promoters from rrnA, rrnB, rrnI, rrnD, rrnE, rrnJ, and rrnO. FIG. 2 also shows the −35 and −10 regions of each promoter, as well as the upstream “UP” elements for each promoter that are upstream of the −35 sequence of each promoter.

FIG. 3 shows the alignment of the rrnE P3 promoter with the P2 promoter from rrnA, rrnB, rrnI, rrnW, rrnH, rrnG, rrnD, rrnJ, and rrnO. FIG. 3 also shows the −35 and −10 regions of each promoter, as well as the upstream “UP” elements for each promoter that are upstream of the −35 sequence of each promoter.

FIG. 4 is a graph showing the cell density measurements for strains expressing GFP from various Papre, PrrnI, PrrnE or PrrnB promoters.

FIG. 5 is a graph showing the cell density measurements for strains expressing FNA from Papre, PrrnI promoters in strain BG8000.

FIG. 6 is a graph showing the cell density measurements for strains expressing FNA from Papre, PrrnI, PrrnE, and PrrnB promoters in strain BG8010.

FIGS. 7A and 7B are graphs showing the cell density measurements for strains expressing ER11 from PaprE and PrrnI promoters in strains BG8000 and BG8010.

FIG. 8 is a graph showing GFP expression from PaprE, PrrnI, PrrnE, and PrrnB promoters.

FIG. 9 is a graph showing FNA expression from PaprE and PrrnI promoters.

FIG. 10 is a graph showing FNA expression from PaprE, PrrnI, PrrnE, and PrrnB promoters.

FIG. 11 is a graph showing ER11 expression from PaprE and PrrnI promoters.

FIG. 12 is a graph showing cell density measurements of strains expressing FNA from PaprE and PrrnI promoters.

FIG. 13 is a graph showing strains expressing FNA from PaprE and PrrnI promoters.

FIG. 14 is a graph showing cell density measurements of FNA expression from single copy integrants of PaprE and PrrnI promoter constructs.

FIG. 15 is a graph showing FNA expression from FNA expression from single copy integrants of PaprE and PrrnI promoter constructs.

FIG. 16 is a graph showing the cell density measurements for strains expressing GFP from various Papre, PrpsJ and PrpsD promoters.

FIG. 17 is a graph showing the cell density measurements for strains expressing FNA from Papre, PrpsD and PrpsJ promoters in strain BG8010.

FIG. 18 is a graph showing GFP expression from Papre, PrpsD and PrpsJ promoters.

FIG. 19 is a graph showing FNA expression from Papre, PrpsD and PrpsJ promoters.

FIG. 20 is a graph showing cell density measurements of strains expressing FNA from PaprE and PrpoD promoters.

FIG. 21 is a graph showing strains expressing FNA from PaprE and PrpoD promoters.

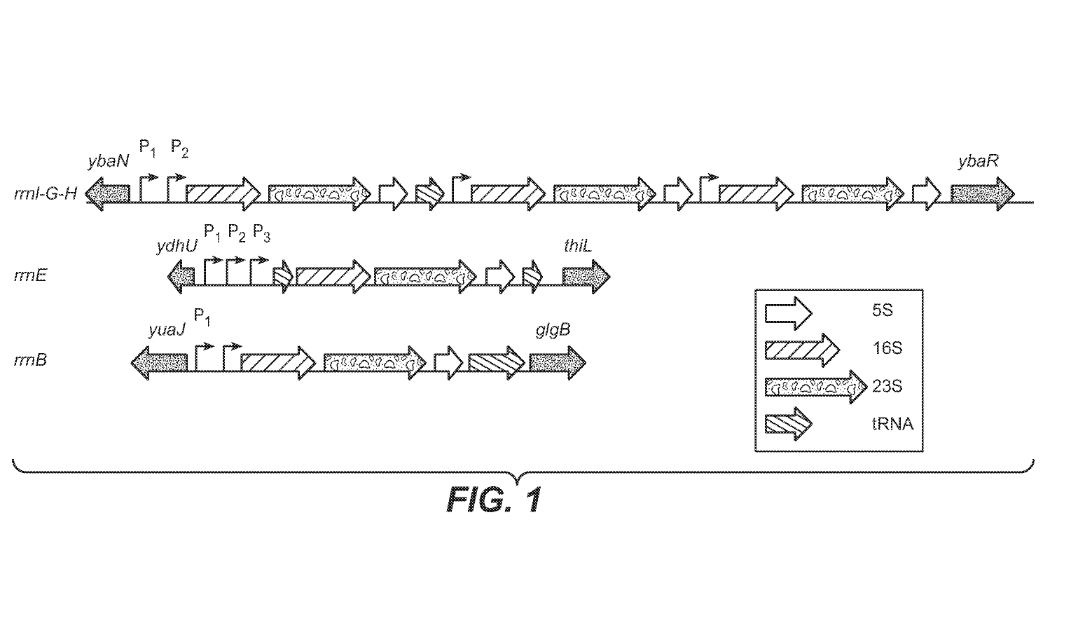

### DETAILED DESCRIPTION OF THE INVENTION

The present invention provides improved methods and compositions for expression systems in microorganisms. In certain embodiments, the methods and compositions comprise a ribosomal promoter derived from a Bacillus species microorganism. Ribosomal promoters include, for example, ribosomal RNA promoters and ribosomal protein promoters. In some embodiments, novel production microorganisms and methods for producing a protein of interest are provided.

All patents and publications, including all sequences disclosed within such patents and publications, referred to herein are expressly incorporated by reference.

### A. Definitions

Unless defined otherwise herein, all technical and scientific terms used herein have the same meaning as commonly understood by one of ordinary skill in the art to which this invention belongs (See e.g., Singleton, et al., DICTIONARY OF MICROBIOLOGY AND MOLECULAR BIOLOGY, 2D ED., John Wiley and Sons, New York [1994], and Hale & Marham, THE HARPER COLLINS DICTIONARY OF BIOLOGY, Harper Perennial, NY [1991]). Although any methods and materials similar or equivalent to those described herein can be used in the practice or testing of the present invention, the preferred methods and materials are described.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

As used herein, the term “nucleic acid sequence” encompasses DNA, RNA, single or doubled stranded and modification thereof. The terms “nucleic acid sequence” and “polynucleotide” may be used interchangeability herein.

As used herein, “polypeptide,” “peptide” and “protein” are used interchangeably and include reference to a polymer of amino acid residues. The terms apply to amino acid polymers in which one or more amino acid residue is an artificial chemical analog of a corresponding naturally occurring amino acid, as well as to naturally occurring amino acid polymers. The terms also apply to polymers containing conservative amino acid substitutions such that the polypeptide remains functional.

As used herein, the term “host cell” refers to a cell that has the capacity to act as a host and expression vehicle for an incoming sequence (i.e., a sequence introduced into the cell), as described herein. In one embodiment, the host cell is a microorganism. In a preferred embodiment, the host cells are Bacillus species.

As used herein, “Bacillus” refers to all species, subspecies, strains and other taxonomic groups within the genus Bacillus, including, but not limited to B. subtilis, B. licheniformis, B. lentus, B. brevis, B. stearothermophilus, B. alcalophilus, B. amyloliquefaciens, B. coagulans, B. circulans, B. lautus, and B. thuringiensis.

As used herein, the term “DNA construct” or “expression construct” refers to a nucleic acid sequence, which comprises at least two DNA polynucleotide fragments. A DNA or expression construct can be used to introduce nucleic acid sequences into a host cell or organism. The DNA may be generated in vitro (e.g., by PCR) or any other suitable techniques. In some preferred embodiments, the DNA construct comprises a sequence of interest. The sequence of interest's nucleic acid is operably linked to a promoter. In some embodiments, the DNA construct further comprises at least one selectable marker. In further embodiments, the DNA construct comprises sequences homologous to the host cell chromosome. In other embodiments, the DNA construct includes non-homologous sequences.

As used herein, the terms “nucleic acid encoding a protein of interest” or “coding sequence of interest” are used interchangeably and mean a nucleic acid sequence that encodes a protein of interest when translated into the protein. In some embodiments, the coding region is present in a cDNA form, while in other embodiments, it is present in genomic DNA or RNA form. When present in a DNA form, the oligonucleotide may be single-stranded (i.e., the sense strand) or double-stranded. In some embodiments, suitable control elements (e.g., enhancers, promoters, splice junctions, polyadenylation signals, etc.) are placed in close proximity to the coding region of the gene if needed to permit proper initiation of transcription and/or correct processing of the primary RNA transcript. Alternatively, in some embodiments, the coding region utilized in the expression vectors of the present invention contain endogenous enhancers, splice junctions, intervening sequences, polyadenylation signals, or a combination of both endogenous and exogenous control elements.

As used herein, the terms “promoter,” “promoter element,” and “promoter sequence,” refer to a DNA sequence which is capable of controlling the transcription of an oligonucleotide sequence into mRNA when the promoter is placed at the 5′ end of (i.e., precedes) an oligonucleotide sequence. Thus, a promoter is typically located 5′ (i.e., upstream) of an oligonucleotide sequence whose transcription into mRNA it controls, and provides a site for specific binding by RNA polymerase and for initiation of transcription. As used herein a ribosomal promoter includes, for example, a ribosomal RNA promoter or a ribosomal protein promoter.

The term “operably linked” refers to juxtaposition, wherein elements are in an arrangement allowing them to be functionally related. For example, a promoter is operably linked to a coding sequence of interest if it controls the transcription of the sequence.

As used herein, the term “promoter activity” when made in reference to a nucleic acid sequence refers to the ability of the nucleic acid sequence to initiate transcription of an oligonucleotide sequence into mRNA.

The term “vector” is defined herein as a polynucleotide designed to carry nucleic acid sequences to be introduced into one or more cell types. Vectors include cloning vectors, expression vectors, shuttle vectors, plasmids, phage or virus particles, DNA constructs, cassettes and the like. Typical expression vectors, which also include plasmids, include regulatory sequences such as promoters, signal sequences, a gene of interest and transcription terminators.

The term “isolated” as defined herein, refers to a compound, protein, cell, nucleic acid sequence, or amino acid that is separated from at least one other compound, protein, cell, nucleic acid sequence, amino acid, or other biological substance with which it is ordinarily associated in its natural source.

As used herein the term “coding region” is defined herein as a nucleic acid sequence that is transcribed into mRNA which is translated into a polypeptide when placed under the control of appropriate control sequences including a promoter. A coding sequence may include cDNA, genomic DNA, synthetic DNA and recombinant DNA.

As used herein, the term “wild-type” gene, gene product, or cell refers to a gene, gene product, or cell which has the characteristics of that gene, gene product, or cell when found in a naturally occurring source. A wild-type gene, gene product, or cell is that which is most frequently observed in a population and is thus designated the “normal” or “wild-type” form. As used herein, the terms “wild-type sequence,” and “wild-type gene” are used interchangeably and refer to a sequence that is native or naturally occurring in a host cell.

In contrast, the term “modified,” “mutant,” or “variant” gene, gene product, or cell refers to a gene, gene product, or cell which displays modifications in sequence and/or functional properties (i.e., altered characteristics) when compared to the wild-type form. Sequence modifications can occur by, for example, substitutions, insertions, deletions, or any other modification that results in an altered sequence or characteristic. It is noted that naturally-occurring mutants can be isolated; these are identified by the fact that they have altered characteristics when compared to the wild-type gene or gene product.

As used herein, the terms “modified sequence” and “modified genes” are used interchangeably and refer to a substitution, insertion, deletion, interruption, or any other modification of naturally occurring nucleic acid sequence. In some embodiments, the expression product of the modified sequence is a truncated protein (e.g., if the modification is a deletion or interruption of the sequence). In some embodiments, the truncated protein retains biological activity. In other embodiments, the expression product of the modified sequence is an elongated protein (e.g., if the modification is an insertion into the nucleic acid sequence). In other embodiments, an insertion results in the production of a truncated protein as the expression product (e.g., if the insertion results in the formation of a stop codon).

As used herein, an “incoming sequence” means a DNA sequence that is introduced into the host cell chromosome or genome. The sequence may encode one or more proteins of interest. The incoming sequence may comprise a promoter operably linked to a sequence encoding a protein of interest. In some embodiments, incoming sequences comprise sequence that is already present in the genome of the cell to be transformed, while in other embodiments, it is not already present in the genome of the cell to be transformed (i.e., in some embodiments, it is homologous, while in other embodiments, it is heterologous sequence).

In some embodiments, the incoming sequence encodes at least one homologous or heterologous protein, including, but not limited to a hormone, enzyme, growth factor, or cytokine. In some preferred embodiments, the incoming sequence encodes at least one enzyme including, but not limited to a protease, cellulase, amylase, xylanase, phytase, mannanase, hemicellulase, carbohydrase, hydrolase, esterase, oxidase (such as phenol oxidase), permease, pullulanase, laccase, lipase, reductase, isomerase, epimerase, tautomerase, transferase, kinase, or phosphatase.

In some embodiments, the incoming sequence encodes a functional wild-type gene or operon, a functional mutant gene or operon, or a non-functional gene or operon.

As used herein, the term “reporter gene” refers to a nucleotide sequence, which is capable of expression in cells and where expression of the reporter confers to cells containing the expressed gene, the ability to be easily detected and measured.

As used herein, the term “flanking sequence,” refers to any sequence that is either upstream or downstream of the sequence being discussed (e.g., for sequences A B C, sequence B is flanked by the A and C sequences). In some embodiments, the incoming sequence is flanked by a homology box on each side.

As used herein, the term “homology box” refers to sequences that are homologous to another nucleic acid sequence. For example, a homology box can be homologous to a nucleic acid sequence in genomic DNA. In such instance, the homology box is useful for directing where in a new construct is integrated into the genomic DNA.

As used herein, the term “homologous recombination” refers to the exchange of DNA fragments between two DNA molecules or paired chromosomes (i.e., during crossing over) at the site of identical nucleotide sequences. In one embodiment, chromosomal integration is accomplished via homologous recombination.

As used herein, the term “heterologous” in general refers to a polynucleotide or polypeptide that does not naturally occur in a host cell, or refers to a polynucleotide or polypeptide that is derived from the same genetic source or species as the host cell, but is in a location that is not native to the heterologous sequence. In some embodiments, a heterologous sequence is a non-host sequence, while in other embodiments, it is a modified sequence, a sequence from a different host cell strain, or a homologous sequence from a different chromosomal location of the host cell.

The terms “transfection” and “transformation” as used herein both refer to methods for introducing DNA into cells.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-CAGT-3′,” is complementary to the sequence “5′-ACTG-3′.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

As used herein, the term “chromosomal integration” refers to the process whereby the incoming sequence is introduced into the chromosome (i.e., genome) of a host cell.

As used herein, the term “selectable marker” refers to the use of any “marker” (i.e., indicator), which indicates the presence or absence of a protein or gene of interest. In some embodiments, the term encompasses genes which encode an enzymatic activity that confers the ability to grow in medium lacking what would otherwise be essential. In other embodiments, a selectable marker confers resistance to an antibiotic or drug upon the cell in which the selectable marker is expressed.

As used herein, the term “signal sequence” or “signal peptide” refers to a sequence of amino acids at the N-terminal portion of a protein, which facilitates the secretion of the mature form of the protein outside the cell. The mature form of the extracellular protein lacks the signal sequence which is cleaved off during the secretion process.

“Amplification” is defined herein as the production of additional copies of a nucleic acid sequence. Amplification of a nucleic acid can be performed by, for example, polymerase chain reaction or other technologies that are well known in the art. As used herein, the term “polymerase chain reaction” (“PCR”) refers to the methods of U.S. Pat. Nos. 4,683,195, 4,683,202, and 4,965,188, all of which are hereby incorporated by reference, which describe a method for increasing the concentration of a segment of a target sequence in a DNA sample (e.g., genomic DNA) without cloning or purification.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; or incorporation of .sup.32P-labeled deoxynucleotide triphosphates, such as dCTP or DATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced. The primer is preferably single stranded for maximum efficiency in amplification, but may alternatively be double stranded.

As used herein, the term “probe” refers to an oligonucleotide, whether occurring naturally as in a purified restriction digest or produced synthetically, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double- or single-stranded DNA at or near a specific nucleotide sequence.

### B. Ribosomal Promoters

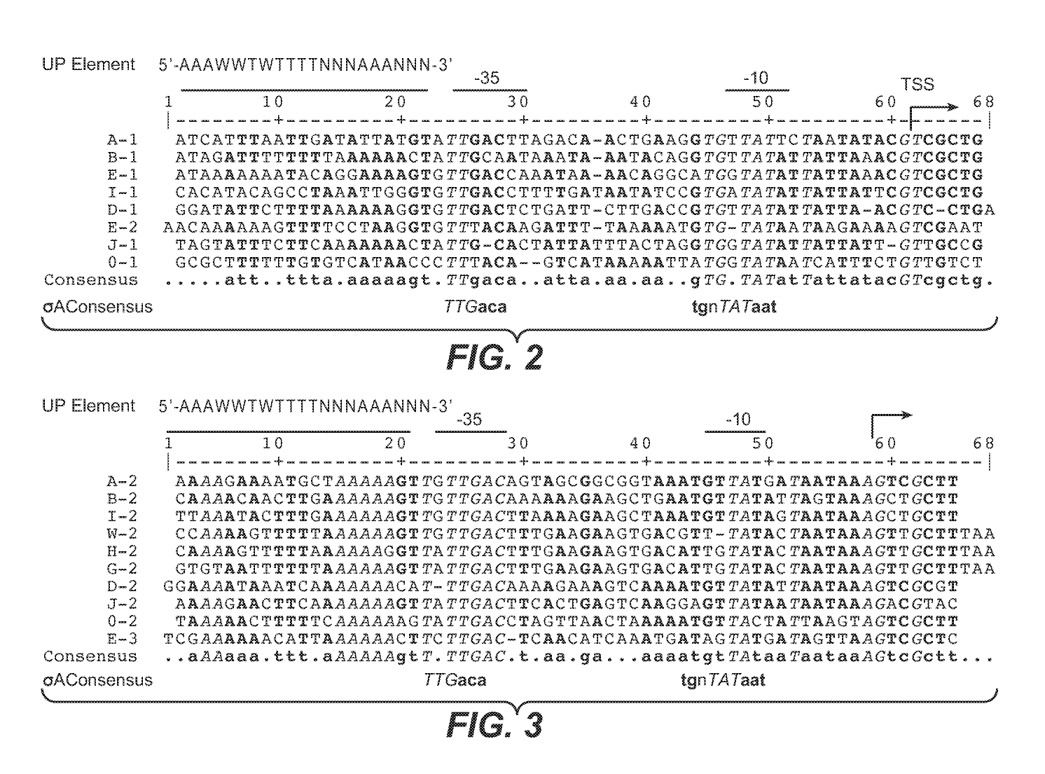

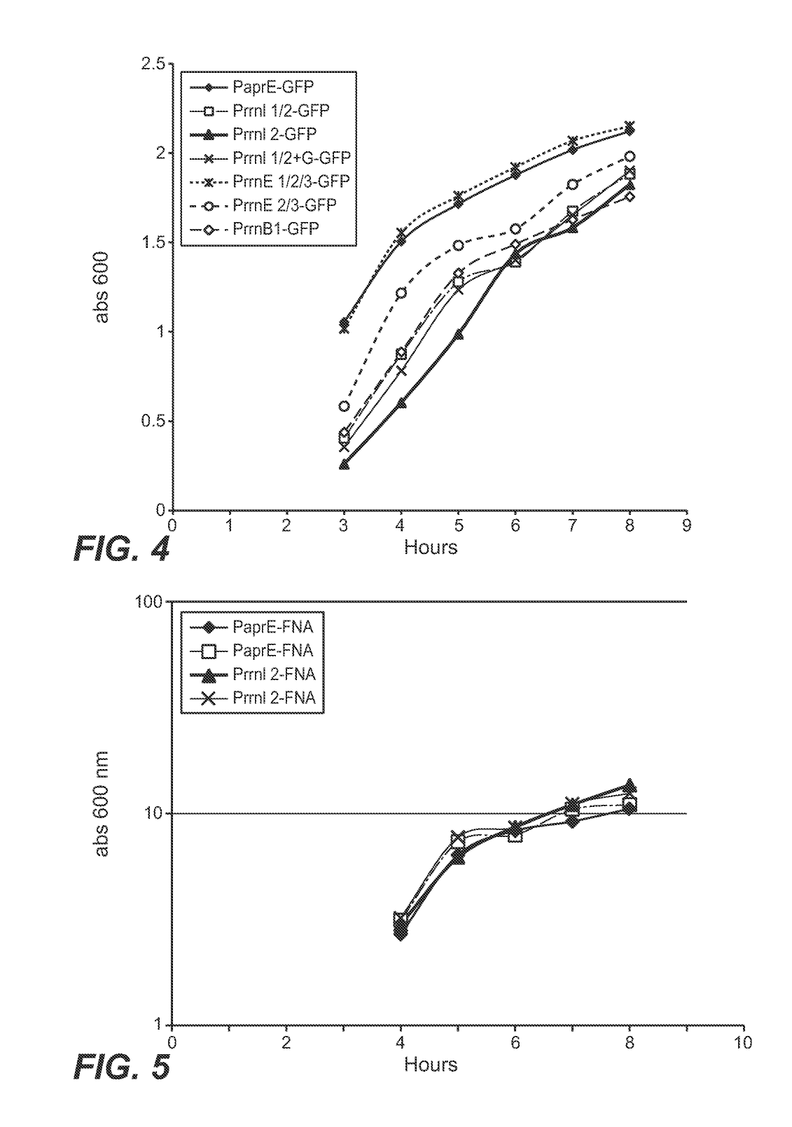

Ribosomal RNA synthesis is the rate-limiting step in ribosome synthesis in Escherichia coli and Bacillus subtilis. The regulation of ribosomal RNA transcription from ribosomal RNA promoters has been studied previously (Samarrai et al., 2011, J Bacteriology, 193:723-733; Natori et al., 2009, J Bacteriology, 191:4555-4561; Turnbough, 2008, Molecular Microbiology 69:10-14; Krasny et al., 2008, Mol Microbiology 69:42-54; Krasny and Gourse, 2004, EMBO 23:4473-4483;). rRNA promoters are tightly regulated with nutritional conditions so that ribosomal RNA and ribosomes are not overproduced in times when translational requirements are lower. In E. coli, there are seven rRNA (rrn) operons, each of which contains two promoters designated P1 and P2. The core −10/−35 region in E. coli rrn P1 promoters is preceded by upstream (UP) elements that increase promoter activity by up to 20-50 fold by binding RNA polymerase. Bacillus subtilis, contains 10 rrn operons (Krasny and Gourse, supra), which are also preceded by upstream (UP) elements that can help to increase promoter activity. See FIGS. 2 and 3.

Although the regulation of ribosomal RNA promoters has been studied for the production of native ribosomal RNAs, the expression levels of a nucleic acid sequence coding for a heterologous protein of interest when using ribosomal RNA promoters has never been investigated.

The regulation of the genes that encode ribosomal proteins has been studied previously in Escherichia coli and Bacillus subtilis (Grundy and Henkin, 1991, J. Bacteriology, 173:4595-4602) In many cases, the ribosomal proteins have been found to act as an autogenous repressor, controlling the expression of the operon in which they are encoded.

The present invention demonstrates that ribosomal promoters, such as ribosomal RNA and protein promoters, are unexpectedly effective at producing heterologous proteins of interest. The amount of mRNA transcribed from a ribosomal promoter was surprisingly high both when compared to other commonly used promoters and as measured by the number of mRNA molecules produced per unit time. See, for example, Examples 3-5 and 9-10 which compare expression from ribosomal promoters to the highly expressed apre promoter. In one embodiment, the ribosomal promoter of the invention provides enhanced transcription efficiency as measured by the number of mRNA molecules produced per unit of time.

The unexpectedly high level of expression of a nucleic acid sequence coding for a heterologous protein of interest when using ribosomal promoters has several benefits. In one embodiment, expressing a coding sequence of interest with a ribosomal promoter allows for increased level of expression of a coding sequence of interest when compared to expression of the coding sequence of interest from its native promoter. An increased level of expression is particularly useful for transcripts that are unstable.

In another embodiment, expressing a coding sequence of interest with a ribosomal promoter allows for increased level of expression of a coding sequence of interest without amplification of an expression construct comprising the ribosomal promoter. When using other expression constructs in the art, in order to achieve high expression levels of a coding sequence of interest, amplification of the expression construct is often required. The expression levels achieved with the ribosomal promoters described herein, however, are high enough that amplification of the expression construct is not necessary. Instead, high expression levels are achieved with a single integrant of the expression construct comprising the ribosomal promoter. See Examples 4 and 5. This provides several benefits. First, host strains are more stable because they do not undergo the loss of the amplified expression construct. Also, if an expression construct does not need to be amplified, strain construction is more efficient. Thus, time, money and materials are saved.

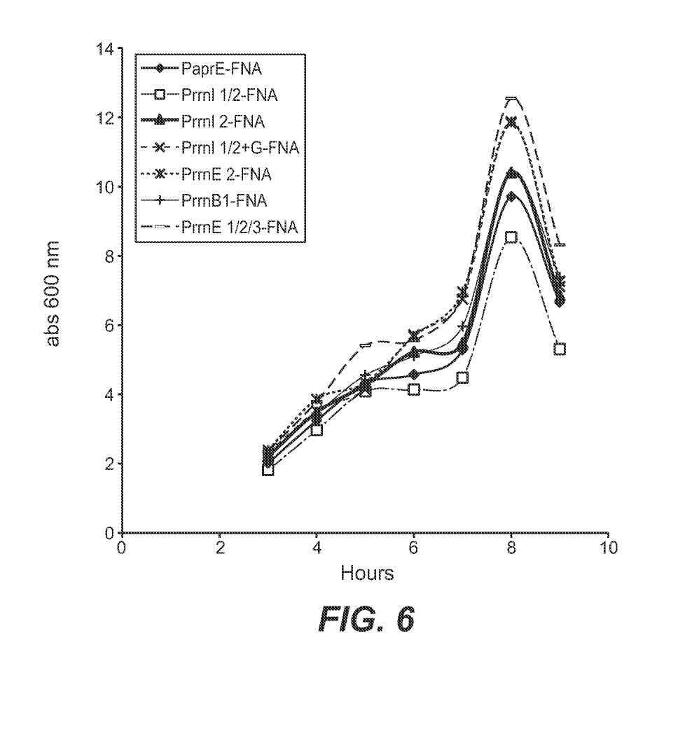

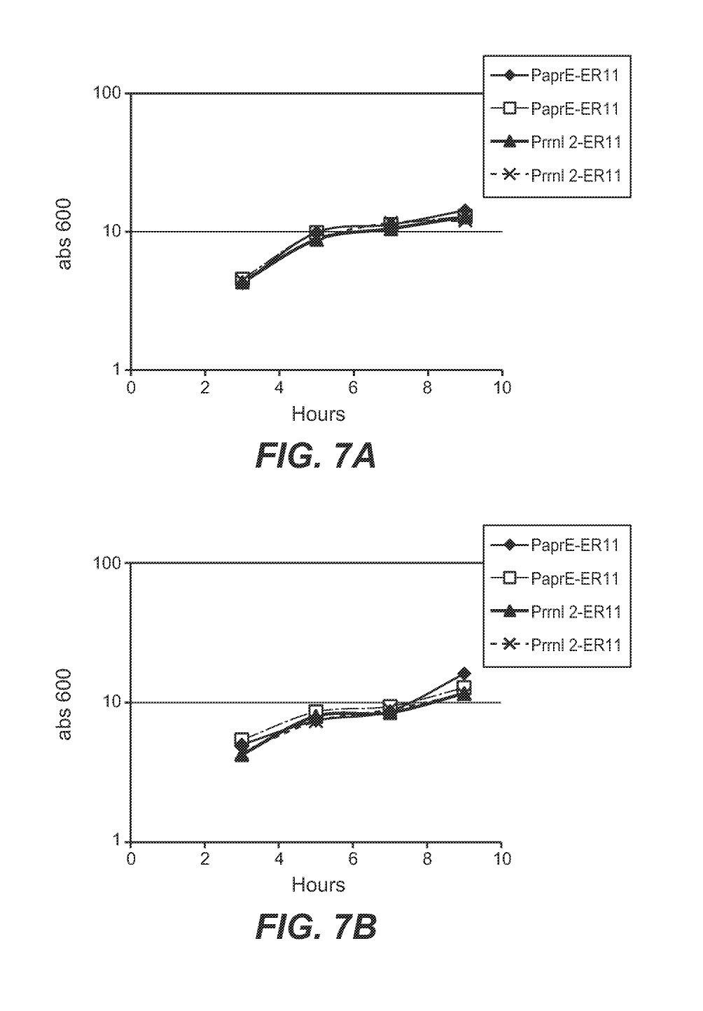

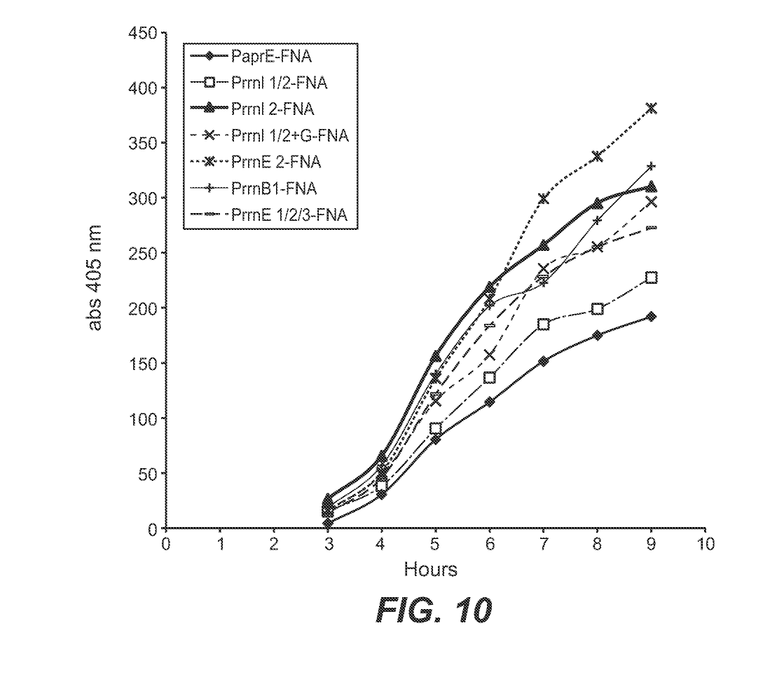

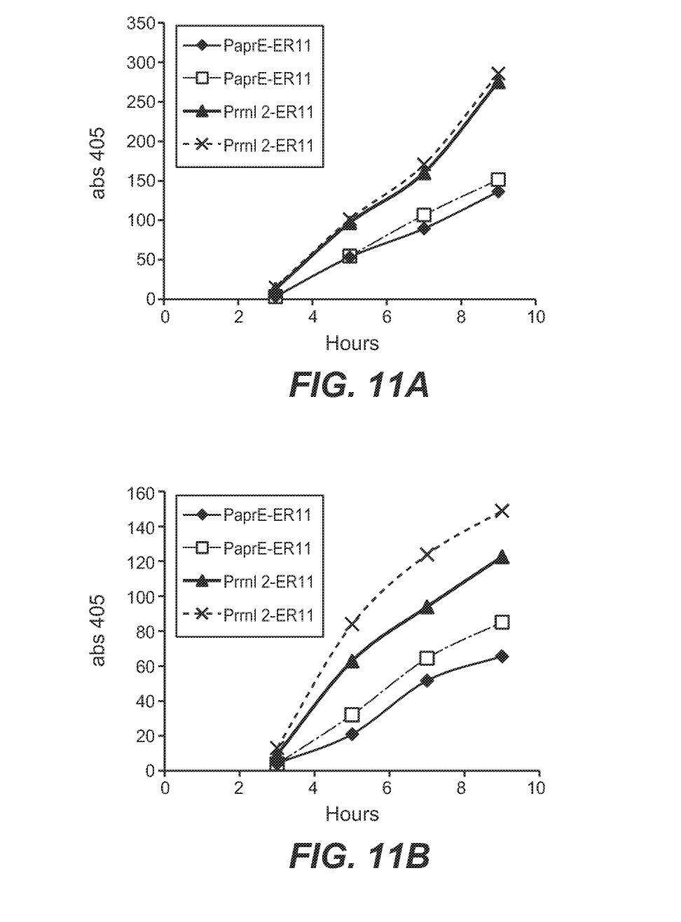

In some embodiments, the ribosomal promoters are ribosomal RNA promoters. The ribosomal RNA promoters used in the invention are any one of the P1, P2, or P3 promoters from a Bacillus rrnI, rrnE, or rrnB ribosomal RNA promoter. In one embodiment, the RNA promoter used in the invention is the P2 promoter from a Bacillus rrnI ribosomal RNA promoter. In some embodiments, combinations of the P1, P2, or P3 promoters from a Bacillus rrnI, rrnE, or rrnB ribosomal RNA promoter can be used. See, for example, Examples 2-4 and FIGS. 4, 8, 6, and 10.

In a particular embodiment, the nucleotide located at the +1 transcriptional start site of a ribosomal promoter (e.g., a ribosomal RNA or protein promoter) described herein is modified from a guanine to adenine. For example, the transcriptional start site for the ribosomal RNA promoters described herein is shown in FIGS. 2 and 3. Modification of the +1 transcriptional start site allows consistent production from a promoter described herein, and therefore, results in better overall productivity from the promoter.

In one embodiment, a promoter has the nucleic acid sequence of any one of SEQ ID NOs: 1-6 or 13-14, or a subsequence thereof. The subsequence will retain promoter activity and comprise at least about 10 nucleotides, at least about 20 nucleotides; at least about 30 nucleotides; at least about 40 nucleotides; at least about 50 nucleotides; at least about 60 nucleotides; at least about 70 nucleotides; at least about 80 nucleotides; at least about 90 nucleotides or at least about 100 nucleotides. The subsequence of any one of SEQ ID NOs: 1-6 or 13-14 should minimally comprise the −35 and −10 regions of the parent promoter. For example, the subsequence of any one of SEQ ID NOs: 1-6 or 13-14 should minimally comprise the −35 and −10 regions of the parent promoter as illustrated in FIGS. 2 and 3, or Tables 1-1 and 2-1. In certain embodiments, a subsequence of any of SEQ ID NOs: 1-6 or 13-14 comprise the −35 and −10 regions of the parent promoter and further comprises the upstream UP elements of the parent promoter, as illustrated in FIGS. 2 and 3.

In a particular embodiment, the promoter has the nucleic acid sequence of SEQ ID NO: 3 or a subsequence thereof. The subsequence will retain promoter activity and comprise at least about 10 nucleotides, at least about 20 nucleotides; at least about 30 nucleotides; at least about 40 nucleotides; at least about 50 nucleotides; at least about 60 nucleotides; at least about 70 nucleotides; at least about 80 nucleotides; at least about 90 nucleotides and at least about 100 nucleotides.

The promoter may also be a hybrid promoter comprising a portion of one or more promoters of the present invention, or a portion of a promoter of the present invention and a portion of another promoter. In some embodiments, the hybrid promoter will include a subsequence of any one of SEQ ID NOs: 1-6 or 13-14 having at least about 10 nucleotides, at least about 20 nucleotides; at least about 30 nucleotides; at least about 40 nucleotides; at least about 50 nucleotides; at least about 60 nucleotides; at least about 70 nucleotides; at least about 80 nucleotides; at least about 90 nucleotides or at least about 100 nucleotides of any one of SEQ ID NOs: 1-6 or 13-14.

The other promoter of the hybrid promoter may be any promoter that shows promoter activity in a host cell, and includes mutant promoters, truncated promoters and the like which may or may not be native to the host cell. Examples of other promoters, which may be useful in a hybrid promoter of the invention, include fungal and bacterial promoters. Some specific nonlimiting examples include; the aprE promoter or a mutant aprE promoter (WO 01/51643); the aph promoter of the Streptomyces fradiae aminoglycoside 3′-phosphotransferase gene; an Aspergillus niger glucoamylase (glaA) promoter; the glucose isomerase (GI) promoter of Actinoplanes missouriensis and the derivative GI (GIT) promoter (U.S. Pat. No. 6,562,612 and EPA 351029); the glucose isomerase (GI) promoter from Streptomyces lividans, the short wild-type GI promoter, the 1.5 GI promoter, the 1.20 GI promoter, or any of the variant GI promoters as disclosed in WO 03/089621; the cbh1, cbh2, eg11 and eg12 promoters from filamentous fungi and specifically the Trichoderma reesei cellobiohydrolase promoter (GenBank Accession No. D86235); the lacZ and tac promoters (Bagdasarion et al., 1983, Gene 26:273-282); the ermE promoter (Ward et al., 1986, Mol. Gen. Genet. 203:468-478 and Schmitt-John et al., 1992, Appl. Microbiol. Biotechnol. 36:493-498); and the Bacillus subtilis phage o29 promoters (Pulido et al., 1986, Gene 49:377-382). Promoters effective in Streptomyces are listed in Hopwood et al., (Hopwood et al., Regulation of Gene Expression in Antibiotic-producing Streptomyces. In Booth, I. and Higgins, C. (Eds) SYMPOSIUM OF THE SOCIETY FOR GENERAL MICROBIOLOGY, REGULATION OF GENE EXPRESSION, Cambridge University Press, 1986 pgs. 251-276). Streptomyces phage promoters are also disclosed in Labes et al., 1997, Microbiol. 143:1503-1512. Other promoters which may be effective for use in the hybrid promoters herein are promoters listed in Deuschle et al., 1986 EMBO J. 5:2987-2994 and WO 96/00787.

The promoter may also be a tandem promoter, which comprises two or more promoters. In some embodiments, the tandem promoter will include the promoter of any one of SEQ ID NOs: 1-6 or 13-14 or a subsequence thereof and one or more other promoters such as those discussed above for hybrid promoters.

A hybrid promoter, a tandem promoter, a promoter which is a subsequence of any one of SEQ ID NOs: 1-6 or 13-14 or a nucleic acid sequence which hybridizes with any one of SEQ ID NOs: 1-6 or 13-14 will have at least about 20%, at least about 30%, at least about 40%, least about 50%, at least about 60%, at least about 80%, and at least about 100% of the promoter activity of its corresponding parent promoter. In some embodiments, the promoter activity will be greater, for example more than about 100%, more than about 150%, more than about 200% and more than about 250%.

In some embodiments, the promoter will include a nucleic acid sequence that hybridizes under medium, high or very high stringency conditions with any one of SEQ ID NOs: 1-6 or 13-14, or a subsequence thereof. In a particular embodiment, the promoter will include a nucleic acid sequence that hybridizes under medium, high or very high stringency conditions with SEQ ID NO: 3, or a subsequence thereof.

In a particular embodiment, hybridization is used to analyze whether a given DNA fragment corresponds to a promoter DNA sequence described herein and thus falls within the scope of the present invention. Sambrook et al., MOLECULAR CLONING: A LABORATORY MANUAL (2.sup.nd Ed., 1989 Cold Spring Harbor, N.Y.) describes general hybridization methods.

“Hybridization conditions” refers to the degree of “stringency” of the conditions under which hybridization is measured. Hybridization conditions can be based on the melting temperature (Tm) of the nucleic acid binding complex, as taught in Berger and Kimmel (1987, Guide to Molecular Cloning Techniques, METHODS IN ENZYMOLOGY, Vol 152, Academic Press, San Diego Calif.). Hybridization conditions can also be based on the washing conditions employed after hybridization as known in the art.

Merely for purposes of illustration, “Low-stringency” conditions can refer to washing with a solution of 0.2×SSC/0.1% SDS at 20 C for 15 minutes. “Medium-stringency” conditions can refer to washing with a solution of 0.2×SSC/0.1% SDS at 37 C for 30 minutes. “High-stringency” conditions can refer to washing with a solution of 0.2×SSC/0.1% SDS at 37 C for 45 minutes. “Very high-stringency” conditions can refer to washing with a solution of 0.2×SSC/0.1% SDS at 37 C for 60 minutes. However, the stringency associated with the particular solution ingredients, temperature, and wash time can vary depending on the particular nucleic acids and other conditions involved. The skilled person would be able to determine the hybridization conditions associated with a desired degree of stringency.

Another aspect of the invention is use of hybridization conditions based on the melting temperature (Tm) of the nucleic acid binding complex, as taught in Berger and Kimmel (1987, Guide to Molecular Cloning Techniques, METHODS IN ENZYMOLOGY, Vol. 152, Academic Press, San Diego, Calif. For purposes of illustration, “very high stringency” typically occurs at about Tm-5 C (5 C below the Tm of the probe); “high stringency” typically occurs at about 5 C to 10 C below Tm; “medium stringency” at about 10 C to 20 C below Tm; and “low stringency” at about 20 C to 25 C below Tm.

One example of a hybridization assay may be performed as follows: Genomic DNA from a particular target source is fragmented by digestion with an appropriate restriction enzyme, e.g., EcoR I, Hind III, Bam HI, Cla I, Kpn I, Mlu I, Spe I, Bgl II, Nco I, Xba I, Xho I and Xma I (supplied by New England Biolabs, Inc., Beverly, Mass. and Boehringer Mannheim) according to the manufacturer's instructions. The samples are then electrophoresed through an agarose gel (for example, 0.8% agarose) so that separation of DNA fragments can be visualized by size. DNA fragments are typically visualized by ethidium bromide staining. The gel may be briefly rinsed in distilled H₂O and subsequently depurinated in an appropriate solution (such as, for example, 0.25M HCl) with gentle shaking followed by denaturation for 30 minutes (m, for example, 0.4 M NaOH) with gentle shaking. A renaturation step may be included, in which the gel is placed in 1.5 M NaCl, 1M Tris, pH 7.0 with gentle shaking for 30 minutes. The DNA should then be transferred onto an appropriate positively charged membrane, for example, Maximum Strength Nytran Plus membrane (Schleicher & Schuell, Keene, N. H.), using a transfer solution (such as, for example, 6×SSC (900 mM NaCl, 90 mM trisodium citrate). Once the transfer is complete, generally after about 2 hours, the membrane is rinsed in e.g., 2×SSC (2×SSC=300 mM NaCl, 30 mM trisodium citrate) and air dried at room temperature. The membrane should then be prehybridized (for approximately 2 hours or more) in a suitable prehybridization solution (such as, for example, an aqueous solution containing per 100 mL: 20-50 mL formamide, 25 mL of 20×SSPE (1×SSPE=0.18 M NaCl, 1 mM EDTA, 10 mM NaH₂PO₄, pH 7.7), 2.5 mL of 20% SDS, and 1 mL of 10 mg/mL sheared herring sperm DNA). As would be known to one of skill in the art, the amount of formamide in the prehybridization solution may be varied depending on the nature of the reaction obtained according to routine methods. Thus, a lower amount of formamide may result in more complete hybridization in terms of identifying hybridizing molecules than the same procedure using a larger amount of formamide. On the other hand, a strong hybridization band may be more easily visually identified by using more formamide.

A DNA probe generally between 50 and 500 bases in length should be isolated by electrophoresis in an agarose gel, the fragment excised from the gel, and recovered from the excised agarose. For a more detailed procedure, see Sambrook, supra. This purified fragment of DNA is then labeled (using, for example, the Megaprime labeling system according to the instructions of the manufacturer) to incorporate P³² in the DNA. The labeled probe is denatured by heating to 95 C for 5 minutes and immediately added to the membrane and prehybridization solution. The hybridization reaction should proceed for an appropriate time and under appropriate conditions, for example, for 18 hours at 37 C with gentle shaking or rotating. The membrane is rinsed (for example, in 2×SSC/0.3% SDS) and then washed in an appropriate wash solution with gentle agitation. The stringency desired will be a reflection of the conditions under which the membrane (filter) is washed.

In one embodiment, the nucleic acid sequence will be the sequence of any one of SEQ ID NOs: 1-6 or 13-14 and the hybridization stringency conditions will be high. In another embodiment, the nucleic acid sequence will be the sequence of SEQ ID NO: 3 and the hybridization stringency conditions will be high.

In other embodiments, a promoter according to the invention will be a subsequence having at least 80%, at least 85%, at least 90%, at least 95%, at least 96%, at least 97%, at least 98% and at least 99% sequence identity with any one of SEQ ID NOs: 1-6 or 13-14. In another embodiment, a promoter according to the invention will be a subsequence having at least 80%, at least 85%, at least 90%, at least 95%, at least 96%, at least 97%, at least 98% and at least 99% sequence identity with SEQ ID NO: 3. A subsequence of any one of SEQ ID NOs: 1-6 or 13-14 should minimally comprise the −35 and −10 regions of the parent promoter, as illustrated in FIGS. 2 and 3 and Tables 1-1 and 2-1. In certain embodiments, a subsequence of any of SEQ ID NOs: 1-6 comprise the −35 and −10 regions of the parent promoter and further comprises the upstream UP elements of the parent promoter, as illustrated in FIGS. 2 and 3.

The term “identity” in the context of two nucleic acid sequences or polypeptides refers to nucleotides or amino acid residues in the two sequences that are the same when aligned for maximum correspondence, as measured using one of the following “sequence comparison algorithms.” Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

An example of an algorithm that is suitable for determining sequence similarity is the BLAST algorithm, which is described in Altschul, et al., J. Mol. Biol. 215:403-410 (1990). Software for performing BLAST analyses is publicly available through the National Center for Biotechnology Information available on the world wide web (www) ncbi.nlm.nih.gov. The BLAST algorithm performs a statistical analysis of the similarity between two sequences (see, e.g., Karlin & Altschul, Proc. Nat'l. Acad. Sci. USA 90:5873-5787 (1993)).

### B. Coding Sequences of Interest

The promoters encompassed by the invention are operably linked to a nucleic acid encoding a protein of interest (i.e., a coding sequence of interest). The polypeptide encoded by the coding sequence may be an enzyme, a hormone, a growth factor, a cytokine, an antibiotic or portion thereof, a receptor or portion thereof, a reporter gene (e.g., green fluorescent protein) or other secondary metabolites.

In some embodiments, the enzyme is a protease, cellulase, hemicellulase, xylanase, amylase, glucoamylase, cutinase, phytase, laccase, lipase, isomerase, esterase, mannanase, carbohydrase, hydrolase, oxidase, permease, pullulanase, reductase, epimerase, tautomerase, transferase, kinase, phosphatase, or the like originating from bacteria or fungi.

In some embodiments, the enzyme is a cellulase. Cellulases are enzymes that hydrolyze the beta-D-glucosidic linkages in celluloses. Cellulolytic enzymes have been traditionally divided into three major classes: endoglucanases, exoglucanases or cellobiohydrolases and beta-glucosidases (Knowles, J. et al., TIBTECH 5:255-261 (1987)). Numerous cellulases have been described in the scientific literature, examples of which include: from Trichoderma reesei: Shoemaker, S. et al., Bio/Technology, 1:691-696, 1983, which discloses CBHI; Teeri, T. et al., Gene, 51:43-52, 1987, which discloses CBHII; Penttila, M. et al., Gene, 45:253-263, 1986, which discloses EGI; Saloheimo, M. et al., Gene, 63:11-22, 1988, which discloses EGII; Okada, M. et al., Appl. Environ. Microbiol., 64:555-563, 1988, which discloses EGIII; Saloheimo, M. et al., Eur. J. Biochem., 249:584-591, 1997, which discloses EGIV; and Saloheimo, A. et al., Molecular Microbiology, 13:219-228, 1994, which discloses EGV Exo-cellobiohydrolases and endoglucanases from species other than Trichoderma have also been described e.g., Ooi et al., 1990, which discloses the cDNA sequence coding for endoglucanase F1-CMC produced by Aspergillus aculeatus; Kawaguchi T et al., 1996, which discloses the cloning and sequencing of the cDNA encoding beta-glucosidase 1 from Aspergillus aculeatus; Sakamoto et al., 1995, which discloses the cDNA sequence encoding the endoglucanase CMCase-1 from Aspergillus kawachii IFO 4308; and Saarilahti et al., 1990 which discloses an endoglucanase from Erwinia carotovara.

In a particular embodiment, the cellulase to be expressed by a promoter of the invention is a cellulase disclosed in U.S. Pat. No. 6,287,839 and U.S. Pat. No. 6,562,612. In certain embodiments, the cellulase to be expressed is a cellulase comprising an amino acid sequence of SEQ ID NO: 1 of U.S. Pat. No. 6,562,612, a fragment or a derivative thereof having cellulolytic activity and greater than 70% sequence identity to an active portion of SEQ ID NO: 1 of U.S. Pat. No. 6,562,612.

In other embodiments, the enzyme is a protease, such as a serine, metallo, thiol or acid protease. In some embodiments, the protease will be a serine protease (e.g., subtilisin). Serine proteases are described in Markland, et al. (1983) Honne-Seyler's Z Physiol. Chem. 364:1537-1540; Drenth, J. et al. (1972) Eur. J. Biochem. 26:177-181; U.S. Pat. Nos. 4,760,025 (RE 34,606), 5,182,204 and 6,312,936 and EP 0 323,299). Proteases that may be used in the invention are also described in, for example, U.S. Patent Publication No. 2010/0152088 and International Publication No. WO 2010/056635. Means for measuring proteolytic activity are disclosed in K. M. Kalisz, “Microbial Proteinases” ADVANCES IN BIOCHEMICAL ENGINEERING AND BIOTECHNOLOGY, A. Fiecht Ed. 1988.

In another embodiment, the protease to be expressed by a promoter of the invention is a protease comprising an amino acid sequence of SEQ ID NOs: 10, 12, 19, or 21, a fragment or a derivative thereof having proteolytic activity and greater than 70% sequence identity to an active portion of SEQ ID NO: 10, 12, 19, or 21. The nucleic acid sequences that encode SEQ ID NOs: 10, 12, 19, or 21 are SEQ ID NOs: 9, 11, 18, and 20, respectively.

In other embodiments, the enzyme is an amylase, such as an amylase derived from Trichoderma (such as T. reesei), a Trichoderma glucoamylase, an amylase derived from Bacillus (such as B. subtilis), or an amylase derived from Geobacillus (such as G. stearothermophilus). Bacterial and fungal amylases are described in, for example, U.S. Pat. No. 8,058,033, U.S. Patent Publication No. 2010/0015686, U.S. Patent Publication No. 2009/0314286, UK application No. 1011513.7, and International Application No. PCT/IB2011/053018. The specifications of each of these references are hereby incorporated by reference in their entirety.

In other embodiments, the enzyme is a xylanase. In certain embodiments, the xylanase is derived from Trichoderma (such as T. reesei). Bacterial and fungal xylanases are described in, for example, International Publication No. WO 2001/027252 and U.S. Pat. No. 7,718,411. The specifications of each of these references are hereby incorporated by reference in their entirety.

In other embodiments, the enzyme is a phytase. In certain embodiments, the phytase is derived from Citrobacter (such as C. freundii) or E. coli. In other embodiments, they phytase may be a Buttiauxella phytase such as a Buttiauxella agrestis phytase. Phytases are described in, for example, International Publication Nos. WO 2006/043178, WO 2006/038062, WO 2008/097619, WO 2009/129489, WO 2006/038128, WO 2008/092901, WO 2009/129489, and WO 2010/122532. The specifications of each of these references are hereby incorporated by reference in their entirety.

In some embodiments, the hormone is a follicle-stimulating hormone, luteinizing hormone, corticotropin-releasing factor, somatostatin, gonadotropin hormone, vasopressin, oxytocin, erythropoietin, insulin and the like.

In some embodiments, the growth factor, which is a protein that binds to receptors on the cell surface with the primary result of activating cellular proliferation and/or differentiation, include platelet-derived growth factor, epidermal growth factor, nerve growth factor, fibroblast growth factor, insulin-like growth factors, transforming growth factors and the like.

In some embodiments, the growth factor is a cytokine. Cytokines include but are not limited to colony stimulating factors, the interleukins (IL-1 (alpha and beta), IL-2 through IL-13) and the interferons (alpha, beta and gamma).

In some embodiments, the antibodies include, but are not limited to, immunoglobulins from any species from which it is desirable to produce large quantities, It is especially preferred that the antibodies are human antibodies. Immunoglobulins may be from any class, i.e. G, A, M, E or D.

The coding sequence may be either native or heterologous to a host cell. In addition, the coding sequence may encode a full-length protein, or a truncated form of a full-length protein. The invention is not limited to a particular coding sequence but encompasses numerous coding sequences, which are operably linked to a promoter of the invention.

### C. Signal Sequences

In some embodiments, especially when the coding sequence of interest codes for an extracellular enzyme, such as a cellulase, protease or starch degrading enzyme, a signal sequence may be linked to the N-terminal portion of the coding sequence. The signal may be used to facilitate the secretion of a DNA sequence. The signal sequence may be endogenous or exogenous to the host organism. The signal sequence may be one normally associated with the encoded polypeptide. In some embodiments, the signal sequence may be altered or modified as described in International Patent Publication Nos. WO 2011/014278 and WO 2010/123754, the specifications of which are hereby incorporated by reference in their entirety. In some embodiments, the signal sequence comprises a signal sequence from a Streptomyces cellulase gene. In one embodiment, a preferred signal sequence is a S. lividans cellulase, celA (Bently et al., (2002) Nature 417:141-147). However, one skilled in the art is aware of numerous signal peptides which may be used depending on a protein to be expressed and secreted in a host organism.

### D. DNA Constructs and Vectors

The nucleic acid construct of the invention comprising a coding region of interest may be prepared synthetically by established standard methods, e.g., the phosphoramidite method described by Beaucage and Caruthers, (1981) Tetrahedron Letters 22:1859-1869, or the method described by Matthes et al., (1984) EMBO Journal 3: 801-805. The nucleic acid construct may be of mixed synthetic and genomic origin and may be prepared by ligating fragments of synthetic or genomic DNA. The nucleic acid construct may also be prepared by polymerase chain reaction using specific primers, for instance as described in U.S. Pat. No. 4,683,202 or Saiki et al., Science 239 (1988), 487-491.

A DNA construct of the invention may be inserted into a vector, such as an expression vector. A variety of vectors suitable for the cloning, transformation and expression of polypeptides in fungus, yeast and bacteria are known by those of skill in the art. Typically, the vector or cassette will comprise a promoter of the invention, optionally a signal sequence, a coding region of interest and a terminator sequence. In preferred embodiments, the vector will include one or more cloning sties located between the signal sequence and the terminator sequences.

### E. Transformation

A vector of the invention will be transformed into a host cell. General transformation techniques are known in the art (Ausubel et al., 1994, CURRENT PROTOCOLS IN MOLECULAR BIOLOGY and Campbell et al., 1989 Curr. Genet. 16:53-56). Some of these general techniques include, but are not limited to the use of a particle or gene gun (biolistics), permeabilization of filamentous fungi cells walls prior to the transformation process (e.g., by use of high concentrations of alkali, e.g., 0.05 M to 0.4 M CaCl₂ or lithium acetate), protoplast fusion, electroporation, or agrobacterium mediated transformation (U.S. Pat. No. 6,255,115) and the treatment of protoplasts or spheroplasts with polyethylene glycol and CaCl.sub.2 is described in Campbell, et al., (1989) Curr. Genet. 16:53-56, 1989 and Penttila, M. et al., (1988) Gene, 63:11-22.

Transformation and expression methods for bacteria are disclosed in Brigidi, DeRossi, Bertarini, Riccardi and Matteuzzi, (1990), FEMS Microbiol. Lett. 55: 135-138. A preferred general transformation and expression protocol for protease deleted Bacillus strains is provided in Ferrari et al., U.S. Pat. No. 5,264,366.

Transformation and expression in Streptomyces can be found in Hopwood et al., GENETIC MANIPULATION OF STREPTOMYCES: A LABORATORY MANUAL, (1985) John Innis Foundation, Norwich UK.

In other embodiments, transformation and expression in Aspergillus and Trichoderma is described in, for example U.S. Pat. No. 5,364,770; U.S. Pat. No. 6,022,725; and Nevalainen et al., 1992, The Molecular Biology of Trichoderma and its Application to the Expression of Both Homologous and Heterologous Genes, in MOLECULAR INDUSTRIAL MYCOLOGY, Eds. Leon and Berka, Marcel Dekker, Inc. pp. 129-148.

### F. Host Cells

Host cells that may be used according to the invention include both bacterial and fungal cells. Preferred fungal host cells include filamentous fungal cells such as Aspergillus and Trichoderma cells. Preferred bacterial host cells include both gram positive and gram negative cells, including Bacillus, Mycobacterium, Actinomyces and Streptomyces cells. Host cells also include, without limitation, E. coli, Pseudomonas spp. (e.g., P. aeruginoa and P. alcaligenes), Streptomyces spp., (e.g., Streptomyces lividans), B. subtilis, B. licheniformis, B. lentus, B. brevis, B. stearothermophilus, B. alkalophilus, B. amyloliquefaciens, B. coagulans, B. circulans, B. lautus, B. megatherium, and B. thuringiensis.

### G. Cell Culture

Host cells and transformed cells can be cultured in conventional nutrient media. The culture media for transformed host cells may be modified as appropriate for activating promoters and selecting transformants. The specific culture conditions, such as temperature, pH and the like, may be those that are used for the host cell selected for expression, and will be apparent to those skilled in the art. In addition, preferred culture conditions may be found in the scientific literature such as Sambrook, (1982) supra; Kieser, T, M J. Bibb, M J. Buttner, K F Chater, and D. A. Hopwood (2000) PRACTICAL STREPTOMYCES GENETICS. John Innes Foundation, Norwich UK; Harwood, et al., (1990) MOLECULAR BIOLOGICAL METHODS FOR BACILLUS, John Wiley and/or from the American Type Culture Collection (ATCC; “http://www.atcc.org/”). Stable transformants of fungal host cells, such as Trichoderma cells can generally be distinguished from unstable transformants by their faster growth rate or the formation of circular colonies with a smooth rather than ragged outline on solid culture medium.

### H. Recovery of Expressed Polypeptides

A polypeptide produced by the transformed host cell may be recovered from the culture medium by conventional procedures including separating the host cells from the medium by centrifugation or filtration, or if necessary, disrupting the cells and removing the supernatant from the cellular fraction and debris. Typically after clarification, the proteinaceous components of the supernatant or filtrate are precipitated by means of a salt, e.g., ammonium sulphate. The precipitated proteins are then solubilized and may be purified by a variety of chromatographic procedures, e.g., ion exchange chromatography, gel filtration chromatography, affinity chromatography, and other art-recognized procedures.

### I. Construct Assembly

In one general embodiment, the present invention involves assembling a DNA construct in vitro, followed by direct cloning of such construct into competent host cells (e.g., Bacillus host cells) such that the construct becomes integrated into the host genome. For example, in some embodiments PCR fusion and/or ligation are employed to assemble a DNA construct in vitro. In a preferred embodiment, the DNA construct is a non-plasmid DNA construct. In another embodiment, the DNA construct comprises a DNA into which a mutation has been introduced. This construct is then used to transform host cells. In this regard, highly competent mutants of a host cell (e.g., Bacillus) are preferably employed to facilitate the direct cloning of the constructs into the cells. For example, Bacillus carrying the comK gene under the control of a xylose-inducible promoter (Pxyl-comK) can be reliably transformed with very high efficiency, as described herein. Any suitable method known in the art may be used to transform the cells. The DNA construct may be inserted into a vector (i.e., a plasmid), prior to transformation. In some preferred embodiments, the circular plasmid is cut using an appropriate restriction enzyme (i.e., one that does not disrupt the DNA construct). Thus, in some embodiments, circular plasmids find use with the present invention. However, in alternative embodiments, linear plasmids are used. In some embodiments, the DNA construct (i.e., the PCR product) is used without the presence of plasmid DNA.

In order to further illustrate the present invention and advantages thereof, the following specific examples are given with the understanding that they are being offered to illustrate the present invention and should not be construed in any way as limiting its scope.

### EXAMPLES

### Example 1

### Generation of Bacillus subtilis Strains Expressing Proteins from Ribosomal RNA and Protein Promoters

The coding sequences of Green fluorescence Protein (GPF), and two subtilisin proteases, FNA (B. amyloliquefaciens subtilisin BPN′-Y217L) and ER11 (described in WO2010/056635A1), were fused to Bacillus subtilis ribosomal RNA or protein promoters to test protein expression in Bacillus subtilis strains BG8000 (ΔnprE, degU(Hy)32, ΔaprE, spoIIE312 amyE::PxylRA-comK-eryR) and BG8010 (ΔnprE, degU(Hy)32, ΔaprE, spoIIE312 amyE::PxylRA-comK-eryR oppA: phleoR). The expression of the proteins from the ribosomal RNA and protein promoters was compared to that obtained from expression with subtilisin promoter aprE (Transcription of Bacillus subtilis subtilisin and expression of subtilisin in sporulation mutants. E Ferrari, D J Henner, M Perego, and J A Hoch, J. Bacteriol. 1988 January; 170(1): 289-295).

The promoters shown in Table 1-1 were amplified by PCR from the Bacillus subtilis 168 chromosomal DNA and transcriptionally fused to the genes for the target molecules (ER11, FNA or GFP). BG8010 or BG8000 strains were transformed with the cassette comprising promoter, gene of interest and antibiotic marker and transformants were selected on LB agar plates containing 5 μg/ml chloramphenicol. BG8010 or BG8000 strains were also transformed with constructs comprising aprE promoter fused to the target molecule genes and transformants were selected on LB agar plates containing 5 μg/ml chloramphenicol. The strains carrying the construct with the subtilisin promoter were amplified on LB agar plates containing 25 μg/ml chloramphenicol to increase the number of copies of the cassette, while the strains carrying the ribosomal promoters were reisolated on plates containing 5 μg/ml chloramphenicol.

TABLE 1-1
List of promoters (the −35 and −10 consensus
sequences are bold and underlined)
Ribosomal RNA Promoters

| | | SEQ ID |
|---|---|---|
| Name | Sequence | NO |
| P1 rrnB | ATAGATTTTTTTTAAAAAACTATTGC | 1 |
| | AATAAATAAATACAGGTGTTATATTA | |
| | TTAAACG TCGCTG | |
| P1 rrnI | CACATACAGCCTAAATTGGGTGTTGA | 2 |
| | CCTTTTGATAATATCCGTGATATATT | |
| | ATTATTCG TCGCTG | |
| P2 rrnI | TTAAATACTTTGAAAAAAGTTGTTGA | 3 |
| | CTTAAAAGAAGCTAAATGTTATAGTA | |
| | ATAAAG CTGCTT | |
| P1 rrnE | ATAAAAAAATACAGGAAAAGTGTTG | 4 |
| | ACCAAATAAAACAGGCATGGTATATT | |
| | ATTAAACG TCGCTG | |
| P2 rrnE | AACAAAAAAGTTTTCCTAAGGTGTTT | 5 |
| | ACAAGATTTTAAAAATGTGTATAATA | |
| | AGAAAAG TCGAAT | |
| P3 rrnE | TCGAAAAAACATTAAAAAACTTCTTG | 6 |
| | ACTCAACATCAAATGATAGTATGATA | |
| | GTTAAG TCGCTC | |

The nucleotide and amino acid sequences of the target molecules are shown below:

Nucleotide sequence of the GFP gene fused to the
ribosomal RNA and protein promoters
(SEQ ID NO: 7)
ACAGAATAGTCTTTTAAGTAAGTCTACTCTGAATTTTTTTAAAAGGAGAG
GGTAAAGAGTGAATAGAAATGTTCTTAAAAATACTGGTCTGAAGGAGATC
ATGTCAGCGAAAGCGTCTGTGGAAGGTATTGTGAACAATCACGTATTCTC
AATGGAGGGGTTTGGAAAGGGAAATGTTTTGTTTGGTAACCAGTTAATGC
AAATTCGAGTTACCAAAGGCGGCCCACTTCCATTTGCCTTCGACATCGTA
AGCATCGCCTTCCAGTACGGCAATCGCACCTTTACGAAATATCCTGATGA
TATCGCCGACTATTTCGTGCAATCGTTTCCAGCGGGCTTTTTCTATGAAA
GAAATCTGCGGTTTGAAGATGGCGCAATCGTTGATATACGTTCAGACATC
AGTCTGGAGGATGACAAGTTTCACTATAAAGTGGAGTATCGAGGAAACGG
ATTTCCGTCTAACGGGCCTGTCATGCAAAAAGCTATTTTGGGCATGGAGC
CGTCTTTTGAAGTGGTTTATATGAATAGCGGCGTCCTTGTAGGGGAAGTG
GATTTAGTTTATAAGCTGGAAAGCGGAAATTATTATTCATGCCATATGAA
AACCTTCTATAGATCAAAGGGCGGAGTGAAAGAATTTCCAGAATATCACT
TTATTCATCATAGACTGGAGAAAACGTATGTTGAAGAAGGTTCTTTCGTC
GAACAGCATGAGACAGCGATCGCTCAGCTTACCACAATAGGCAAACCGCT
GGGTTCGCTCCATGAATGGGTTTAA
Amino acid sequence of the GFP expressed from the
ribosomal RNA and protein promoters
(SEQ ID NO: 8)
VNRNVLKNTGLKEIMSAKASVEGIVNNHVFSMEGFGKGNVLFGNQLMQIR
VTKGGPLPFAFDIVSIAFQYGNRTFTKYPDDIADYFVQSFPAGFFYERNL
RFEDGAIVDIRSDISLEDDKFHYKVEYRGNGFPSNGPVMQKAILGMEPSF
EVVYMNSGVLVGEVDLVYKLESGNYYSCHMKTFYRSKGGVKEFPEYHFIH
HRLEKTYVEEGSFVEQHETAIAQLTTIGKPLGSLHEWV
Nucleotide sequence of the FNA subtilisin
protease gene fused to the ribosomal RNA
and protein promoters
(SEQ ID NO: 9)
ACAGAATAGTCTTTTAAGTAAGTCTACTCTGAATTTTTTTAAAAGGAGAG
GGTAAAGAGTGAGAAGCAAAAAATTGTGGATCAGTTTGCTGTTTGCTTTA
GCGTTAATCTTTACGATGGCGTTCGGCAGCACATCCTCTGCCCAGGCGGC
AGGGAAATCAAACGGGGAAAAGAAATATATTGTCGGGTTTAAACAGACAA
TGAGCACGATGAGCGCCGCTAAGAAGAAAGATGTCATTTCTGAAAAAGGC
GGGAAAGTGCAAAAGCAATTCAAATATGTAGACGCAGCTTCAGCTACATT
AAACGAAAAAGCTGTAAAAGAATTGAAAAAAGACCCGAGCGTCGCTTACG
TTGAAGAAGATCACGTAGCACATGCGTACGCGCAGTCCGTGCCTTACGGC
GTATCACAAATTAAAGCCCCTGCTCTGCACTCTCAAGGCTACACTGGATC
AAATGTTAAAGTAGCGGTTATCGACAGCGGTATCGATTCTTCTCATCCTG
ATTTAAAGGTAGCAGGCGGAGCCAGCATGGTTCCTTCTGAAACAAATCCT
TTCCAAGACAACAACTCTCACGGAACTCACGTTGCCGGCACAGTTGCGGC
TCTTAATAACTCAATCGGTGTATTAGGCGTTGCGCCAAGCGCATCACTTT
ACGCTGTAAAAGTTCTCGGTGCTGACGGTTCCGGCCAATACAGCTGGATC
ATTAACGGAATCGAGTGGGCGATCGCAAACAATATGGACGTTATTAACAT
GAGCCTCGGCGGACCTTCTGGTTCTGCTGCTTTAAAAGCGGCAGTTGATA
AAGCCGTTGCATCCGGCGTCGTAGTCGTTGCGGCAGCCGGTAACGAAGGC
ACTTCCGGCAGCTCAAGCACAGTGGGCTACCCTGGTAAATACCCTTCTGT
CATTGCAGTAGGCGCTGTTGACAGCAGCAACCAAAGAGCATCTTTCTCAA
GCGTAGGACCTGAGCTTGATGTCATGGCACCTGGCGTATCTATCCAAAGC
ACGCTTCCTGGAAACAAATACGGCGCGTTGAACGGTACATCAATGGCATC
TCCGCACGTTGCCGGAGCGGCTGCTTTGATTCTTTCTAAGCACCCGAACT
GGACAAACACTCAAGTCCGCAGCAGTTTAGAAAACACCACTACAAAACTT
GGTGATTCTTTCTACTATGGAAAAGGGCTGATCAACGTACAGGCGGCAGC
TCAGTAA
Amino acid sequence of the FNA subtilisin protease
expressed from the ribosomal RNA and protein
promoters
(SEQ ID NO: 10)
VRSKKLWISLLFALALIFTMAFGSTSSAQAAGKSNGEKKYIVGFKQTMST
MSAAKKKDVISEKGGKVQKQFKYVDAASATLNEKAVKELKKDPSVAYVEE
DHVAHAYAQSVPYGVSQIKAPALHSQGYTGSNVKVAVIDSGIDSSHPDLK
VAGGASMVPSETNPFQDNNSHGTHVAGTVAALNNSIGVLGVAPSASLYAV
KVLGADGSGQYSWIINGIEWAIANNMDVINMSLGGPSGSAALKAAVDKAV
ASGVVVVAAAGNEGTSGSSSTVGYPGKYPSVIAVGAVDSSNQRASFSSVG
PELDVMAPGVSIQSTLPGNKYGALNGTSMASPHVAGAAALILSKHPNWTN
TQVRSSLENTTTKLGDSFYYGKGLINVQAAAQ
Nucleotide sequence of the ER11 subtilisin
protease gene fused to the ribosomal RNA and
protein promoters
(SEQ ID NO: 11)
GAATAGTCTTTTAAGTAAGTCTACTCTGAATTTTTTTAAAAGGAGAGGGT
AAAGAGTGAGAAGCAAAAAATTGTGGATCAGCTTGTTGTTTGCGTTAACG
TTAATCTTTACGATGGCGTTCAGCAACATGTCTGCGCAGGCTGCTGAAGA
AGCAAAAGAAAAATATTTAATTGGCTTTAATGAGCAGGAAGCTGTCAGTG
AGTTTGTAGAACAAGTAGAGGCAAATGACGGCGTCGCCATTCTCTCTGAG
GAAGAGGAAGTCGAAATTGAATTGCTTCATGAATTTGAAACGATTCCTGT
TTTATCCGTTGAGTTAAGCCCAGAAGATGTGGACGCGCTTGAACTCGATC
CAGCGATTTCTTATATTGAAGAGGATGCAGAAGTAACGACAATGGCGCAA
TCGGTACCATGGGGAATTAGCCGTGTGCAAGCCCCAGCTGCCCATAACCG
TGGATTGACAGGTTCTGGTGTAAAAGTTGCTGTCCTCGATACAGGTATTT
CCACTCATCCAGACTTAAATATTCGTGGTGGCGCTAGCTTTGTACCAGGG
GAACCATCCACTCAAGATGGGAATGGGCATGGCACGCATGTGGCTGGGAC
GATTGCTGCTTTAAACAATTCGATTGGCGTTCTTGGCGTAGCACCGAACG
CGGAACTATACGCTGTTAAAGTATTAGGGGCGAGCGGTATGGGTTCGGTC
AGCTCGATTGCCCAAGGATTGGAATGGGCAGGGAACAATGTTATGCACGT
TGCTAATTTGAGTTTAGGACTGCAGGCACCAAGTGCCACACTTGAGCAAG
CTGTTAATAGCGCGACTTCTAGAGGCGTTCTTGTTGTAGCGGCATCTGGC
AATTCAGGTGCAGGCTCAATCAGCTATCCGGCCCGTTATGCGAACGCAAT
GGCAGTCGGAGCTACTGACCAAAACAACAACCGCGCCAGCTTTTCACAGT
ATGGCGCAGGGCTTGACATTGTCGCACCAGGTGTAAACGTGCAGAGCACA
TACCCAGGTTCAACGTATGCCAGCTTAAACGGTACATCGATGGCTACTCC
TCATGTTGCAGGTGCAGCAGCCCTTGTTAAACAAAAGAACCCATCTTGGT
CCAATGTCCAAATCCGCAATCATCTAAAGAATACGGCAACGAGCTTAGGA
AGCACGAACTTGTATGGAAGCGGACTTGTCAATGCAGAAGCGGCAACACG
TTAA
Amino acid sequence of the ER11 subtilisin
protease expressed from the ribosomal RNA
and protein promoters
(SEQ ID NO: 12)
VRSKKLWISLLFALTLIFTMAFSNMSAQAAEEAKEKYLIGFNEQEAVSEF
VEQVEANDGVAILSEEEEVEIELLHEFETIPVLSVELSPEDVDALELDPA
ISYIEEDAEVTTMAQSVPWGISRVQAPAAHNRGLTGSGVKVAVLDTGIST
HPDLNIRGGASFVPGEPSTQDGNGHGTHVAGTIAALNNSIGVLGVAPNAE
LYAVKVLGASGMGSVSSIAQGLEWAGNNVMHVANLSLGLQAPSATLEQAV
NSATSRGVLVVAASGNSGAGSISYPARYANAMAVGATDQNNNRASFSQYG
AGLDIVAPGVNVQSTYPGSTYASLNGTSMATPHVAGAAALVKQKNPSWSN
VQIRNHLKNTATSLGSTNLYGSGLVNAEAATR

The different strains constructed from the fusion of the promoters to the target genes are listed in Table 1-2 below. 1/2/3 indicates that the three promoters were cloned in tandem to drive the expression of the target molecule. The mark “+G” indicates the use of the nucleotide guanine as transcription start site instead of adenine.

TABLE 1-2
List of strains
constructed

BG8000 PaprE-FNA
BG8000 PaprE-FNA
BG8000 PrrnI 2-FNA
BG8000 PrrnI 2-FNA
BG8010 PaprE-FNA
BG8010 PaprE-FNA
BG8010 PrrnI 2-FNA
BG8010 PrrnI 2-FNA
BG8000 PrrnI2-ER11
BG8000 PrrnI 2-ER11
BG8010 PrrnI 2-ER11
BG8010 PrrnI 2-ER11
BG8000 PaprE-ER11
BG8000 PaprE-ER11
BG8010 PaprE-ER11
BG8010 PaprE-ER11
BG8010 PrrnI 1/2-FNA
BG8010 PrrnI 2-FNA
BG8010 PrrnI 1/2 + G-FNA
BG8010 PrrnE 2-FNA
BG8010 PrrnB1-FNA
BG8010 PrrnE 1/2/3-FNA
BG8010 PrrnI 1/2-GFP
BG8010 PrrnI 2-GFP
BG8010 PrrnI 1/2 + G-GFP
BG8010 PrrnE 1/2/3-GFP
BG8010 PrrnE 2/3-GFP
BG8010 PrrnB1-GFP
BG8010 PaprE-GFP

### Example 2

### Cell Density Measurements of GFP, FNA and ER11 Expressing Strains

To test for cell growth, one colony each of the constructed strains was inoculated in Luria Broth containing 5 μg/ml chloramphenicol (for strains expressing from ribosomal RNA promoters) or 25 μg/mlchloramphenicol (for strains expressing from aprE promoters) and grown overnight at 30° C. One ml of each pre-culture was used to inoculate 32 ml of 2×SNB medium (see composition below) and grown at 37° C. in shake flasks at 280 rpm, 70% humidity. At hourly intervals from 4 hours to 8 hours of growth, optical densities of each culture was measured at 600 nm using a SpectraMax reader. The cell density measurements of GFP, FNA, and ER11 expressing strains are shown in FIGS. 4 (GFP), 5 and 6 (FNA), and 7A and 7B (ER11). The growth of strains containing the different constructs was comparable.

2×SNB Medium:
Stock solutions (filter sterilized): 25×SNB salts-CaCl₂*2H₂O (3.7 g/L), FeSO₄*7H₂O (9.6 mg/L), MnCl₂*4H₂O (6 mg/L), KCl (25 g/L), MgSO₄*7H₂O (3.26 g/L), Maltrin 150 10% Prepare 500 mL of 16 g/L solution of Difco Nutrient Broth, autoclave, add 20 mL 25×SNB salts, and 25 mL 10% Maltrin 150.

### Example 3

### Protein Expression of GFP, FNA, and ER11 from Ribosomal Promoters

The extracellular production of ER11, FNA or intracellular expression of GFP driven by the selected promoters was tested in BG8000 and BG8010 strains. The cells were grown as described for the cell density measurements in Example 2. At hourly intervals from 4 hours to 8 hours of growth, supernatants of cultures were analyzed for AAPF activity (subtilisin expression). GFP expression was measured as Relative Fluorescence Units (RFU) expressed in the cell.

The AAPF activity of a sample was measured as the rate of hydrolysis of N-succinyl-L-alanyl-L-alanyl-L-prolyl-L-phenyl-p-nitroanilide (suc-AAPF-pNA). The reagent solutions used were: 100 mM Tris/HCl, pH 8.6, containing 0.005% TWEEN®-80 (Tris dilution buffer and 160 mM suc-AAPF-pNA in DMSO (suc-AAPF-pNA stock solution) (Sigma: S-7388). To prepare a suc-AAPF-pNA working solution, 1 ml suc-AAPF-pNA stock solution was added to 100 ml Tris/HCl buffer and mixed well for at least 10 seconds. The assay was performed by diluting the samples in the assay buffer (5 μl in 195 μl). Then, 180 μl of assay buffer with AAPF substrate was added to 20 μl of the diluted sample arrayed in a microtiter plate. The solutions were mixed for 5 sec., and the absorbance change in kinetic mode (20 readings in 5 minutes) was read at 405 nm in a SpectraMax reader, at 25° C.

For measuring GFP expression in RFU, 150 μl of each culture sample was loaded into a microtiter plate and fluorescence measurements were taken using the SpectraMax reader using an excitation wavelength at 485 nm, emission wavelength at 508 nm, with a cutoff at 495 nm.

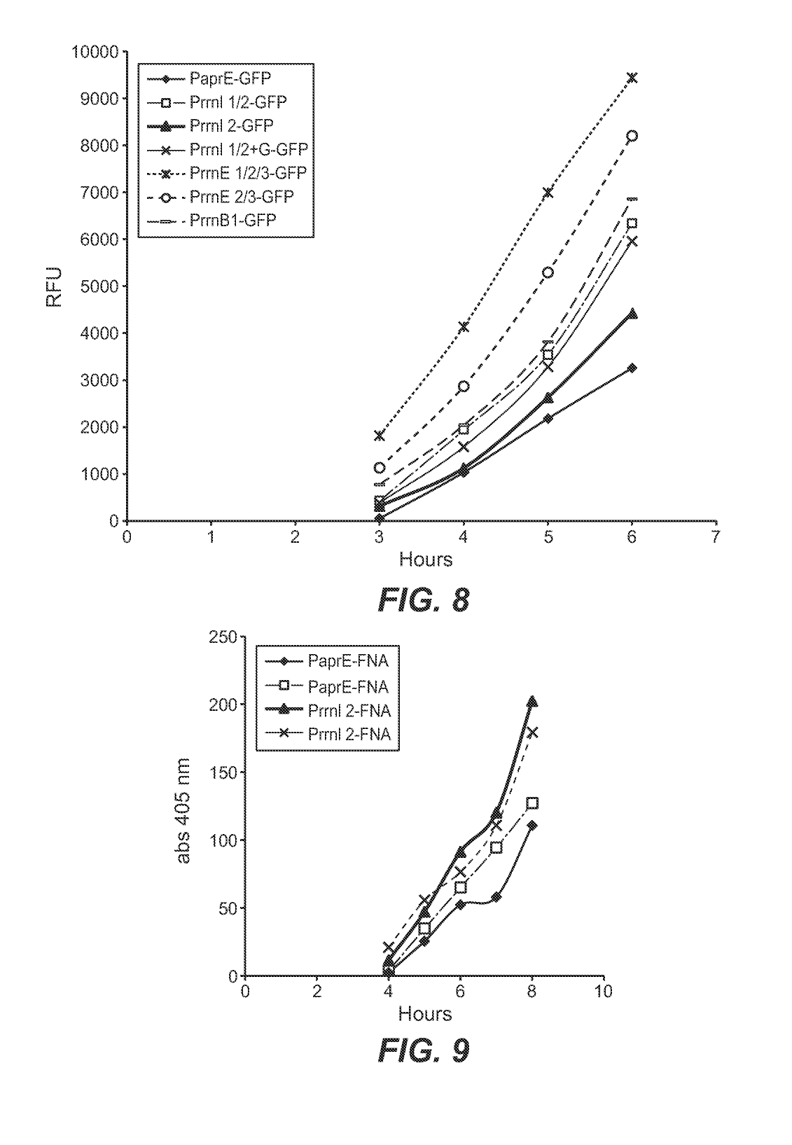

Expression of GFP, FNA, and ER11 from the different promoters is shown in FIGS. 8 (GFP), 9 and 10 (FNA), and 11A and 11B (ER11). Protein expression from non-amplified ribosomal RNA promoter and protein promoter was higher than that seen from amplified aprE promoter.

### Example 4

### Protein Expression from SigmaA Dependent Promoter

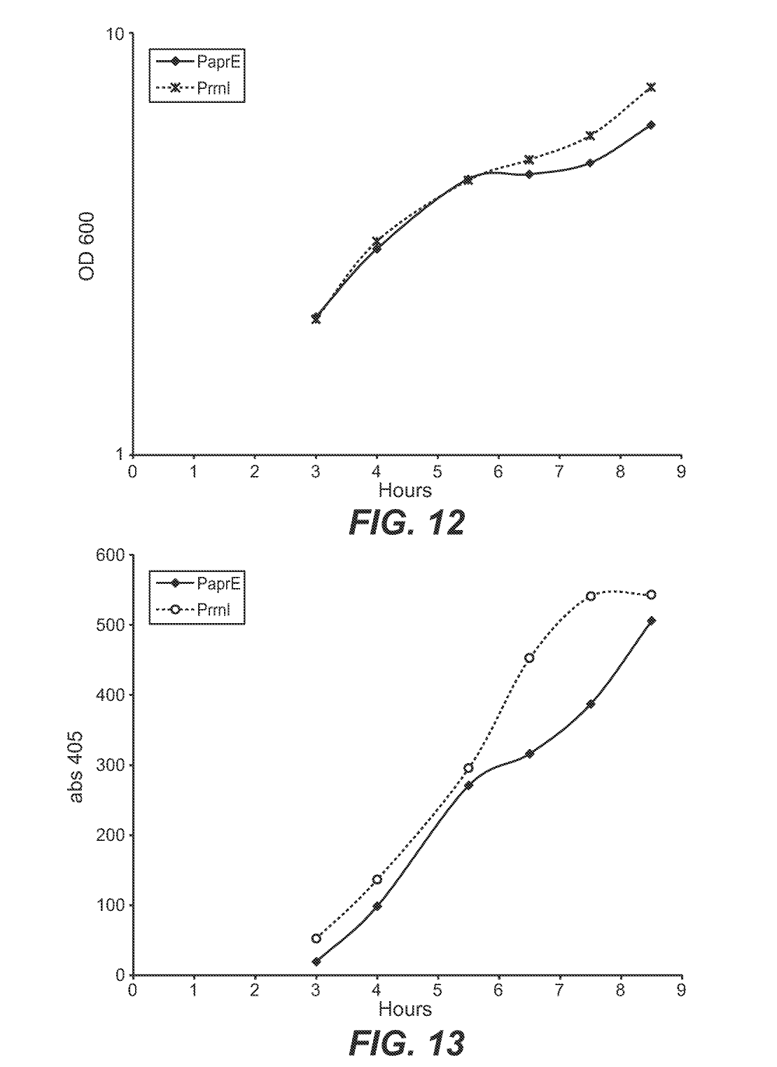

As different levels of protein expression are observed from different promoters, this experiment compared FNA expression amplified aprE promoter and unamplified rrnI P2 promoter. BG8010 strains expressing FNA from aprE were amplified using 25 μg/mL chloramphenicol, while strains expressing FNA from rrnI P2 were reisolated on 5 μg/mL chloramphenicol as described in Example 1. Cell density measurements and FNA expression was studied as described in Examples 2 and 3 respectively. Results are shown in FIGS. 12 and 13. Cell growth from all strains was comparable, but FNA expression from unamplified rrnI P2 promoter was higher that from amplified aprE promoters.

### Example 5

### FNA Expression from BG8010 Strains Containing Single Copy Integrant

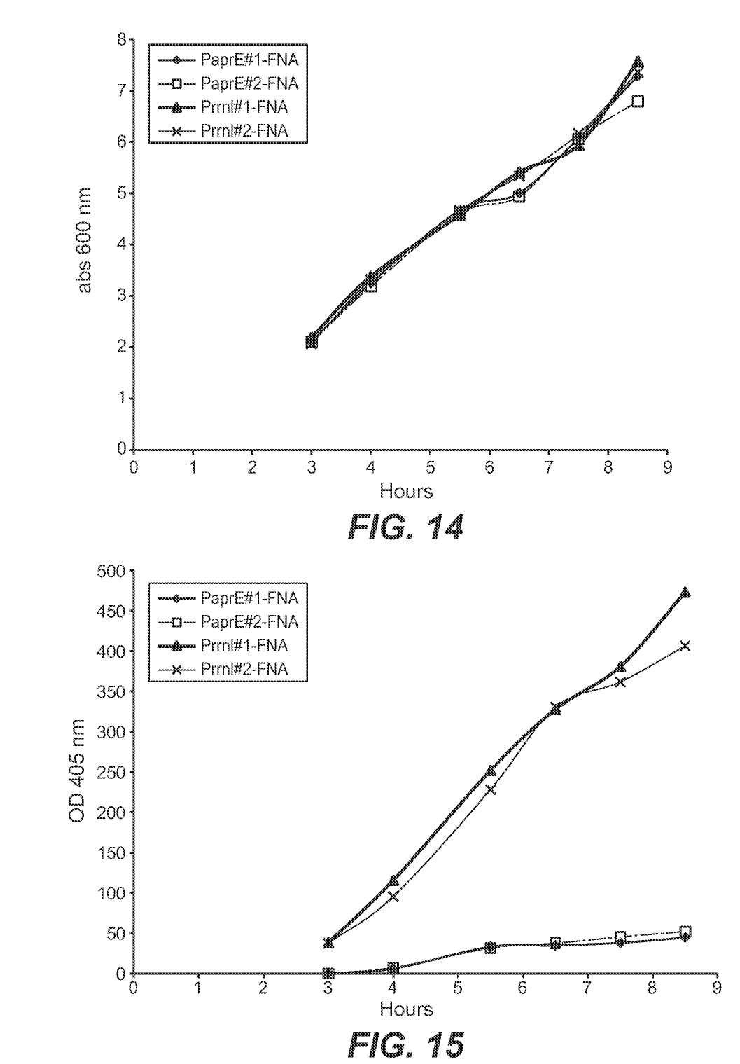

To test whether rrnI P2 promoter could be used for protein expression without the use of antibiotic marker, a single copy integrant containing either PrrnI P2-FNA SpcR or PaprE-FNA CatR cassette was integrated in the BG8010 strain by double cross over integration. The antibiotic marker genes flanked by lox sequences were subsequently removed using cre-lox recombinase. Transformants of constructed strains were grown as described in Example 2 and cell density measurements and FNA expression were studied as described in Example 2 and 3 respectively. Results shown in FIG. 14 indicate that growth of strains containing either the rrnI P2 or aprE promoter was comparable, but FNA expression from PrrnI-P2 was higher than from aprE (FIG. 15). These studies demonstrate that PrrnI-P2 is a strong promoter that can deliver high amount of mRNA of the target molecule. The advantage of using this promoter consists in delivering high amount of transcript without the need of the amplification of the construct and without the use of the antibiotic marker.

### Example 6

### Generation of Bacillus subtilis Strains Expressing Proteins from Ribosomal Protein Promoters

The coding sequences of Green fluorescence Protein (GPF), and two subtilisin proteases, FNA (B. amyloliquefaciens subtilisin BPN′-Y217L) and ER11 (described in WO2010/056635A1), were fused to Bacillus subtilis ribosomal protein promoters to test protein expression in Bacillus subtilis strains BG8000 (ΔnprE, degU(Hy)32, ΔaprE, spoIIE312 amyE::PxylRA-comK-eryR) and BG8010 (ΔnprE, degU(Hy)32, ΔaprE, spoIIE312 amyE::PxylRA-comK-eryR oppA: phleoR). The expression of the proteins from the ribosomal protein promoters was compared to that obtained from expression with subtilisin promoter aprE (Transcription of Bacillus subtilis subtilisin and expression of subtilisin in sporulation mutants. E Ferrari, D J Henner, M Perego, and J A Hoch, J. Bacteriol. 1988 January; 170(1): 289-295).

The promoters shown in Table 2-1 were amplified by PCR from the Bacillus subtilis 168 chromosomal DNA and transcriptionally fused to the genes for the target molecules (FNA or GFP). BG8010 or BG8000 strains were transformed with the cassette comprising promoter, gene of interest and antibiotic marker and transformants were selected on LB agar plates containing 5 μg/mlchloramphenicol. BG8010 or BG8000 strains were also transformed with constructs comprising aprE promoter fused to the target molecule genes and transformants were selected on LB agar plates containing 5 μg/mlchloramphenicol. The strains carrying the construct with the subtilisin (aprE) promoter were amplified on LB agar plates containing 25 μg/mlchloramphenicol to increase the number of copies of the cassette, while the strains carrying the ribosomal promoters were reisolated on plates containing 5 μg/ml chloramphenicol.

TABLE 2-1
List of promoters (the −35 and −10 consensus
sequences are bold and underlined)

| | | |
|---|---|---|
| | | SEQ ID |
| Name | Sequence | NO |

Ribosomal protein promoters

| | | |
|---|---|---|
| rpsD | GTTTTTATCACCTAAAAGTTTACCACT | 13 |
| | AATTTTTGTTTATTATATCATAAACGG | |
| | TGAAGCAATAATGGAGGAATGGTTGA | |
| | CTTCAAAACAAATAAATTATATAATG | |
| | ACCTTT | |
| rpsJ | GTACCGTGTGTTTTCATTTCAGGGAAA | 14 |
| | CATGACTTAATTGTTCCTGCAGAAATA | |
| | TCGAAACAGTATTATCAAGAACTTGA | |
| | GGCACCTGAAAAGCGCTGGTTTCAAT | |
| | TTGAGAATTCAGCTCACACCCCGCAT | |
| | ATTGAGGAGCCATCATTATTCGCGAA | |
| | CACATTAAGTCGGCATGCACGCAACC | |
| | ATTTATGATAGATCCTTGATAAATAA | |
| | GAAAAACCCCTGTATAATAAAAAAA | |
| | GTGTGCAAATGATGCATATTTTAAAT | |
| | AAGTCTTGCAACATGCGCCTATTTTCT | |
| | GTATAATGGTGTATA | |

Sigma factor promoter

| | | |
|---|---|---|
| rpoD (P1) | AACATATAACTCAGGACGCTCTATCC | 15 |
| | TGGGTTTTTGGCTGTGCCAAAAGGGA | |
| | ATAATGAAAAACAATAGCATCTTTGT | |
| | GAAGTTTGTATTATAATAAAAAATT | |

Table 2-1: Promoter sequences are shown for rpsD, rpsJ, and rpoD (P1). −35 and −10 sequences are shown in bold and underlined for each promoter. For rpsJ, two promoters are available (P1 and P2). The −35 and −10 sequences for rpsJ P1 are upstream (i.e., 5′) of the −35 and −10 sequences for rpsJ P2 sequences.

Nucleotide sequence of the GFP gene fused to the
ribosomal protein promoters
(SEQ ID NO: 16)
ACAGAATAGTCTTTTAAGTAAGTCTACTCTGAATTTTTTTAAAAGGAGAG
GGTAAAGAGTGAATAGAAATGTTCTTAAAAATACTGGTCTGAAGGAGATC
ATGTCAGCGAAAGCGTCTGTGGAAGGTATTGTGAACAATCACGTATTCTC
AATGGAGGGGTTTGGAAAGGGAAATGTTTTGTTTGGTAACCAGTTAATGC
AAATTCGAGTTACCAAAGGCGGCCCACTTCCATTTGCCTTCGACATCGTA
AGCATCGCCTTCCAGTACGGCAATCGCACCTTTACGAAATATCCTGATGA
TATCGCCGACTATTTCGTGCAATCGTTTCCAGCGGGCTTTTTCTATGAAA
GAAATCTGCGGTTTGAAGATGGCGCAATCGTTGATATACGTTCAGACATC
AGTCTGGAGGATGACAAGTTTCACTATAAAGTGGAGTATCGAGGAAACGG
ATTTCCGTCTAACGGGCCTGTCATGCAAAAAGCTATTTTGGGCATGGAGC
CGTCTTTTGAAGTGGTTTATATGAATAGCGGCGTCCTTGTAGGGGAAGTG
GATTTAGTTTATAAGCTGGAAAGCGGAAATTATTATTCATGCCATATGAA
AACCTTCTATAGATCAAAGGGCGGAGTGAAAGAATTTCCAGAATATCACT
TTATTCATCATAGACTGGAGAAAACGTATGTTGAAGAAGGTTCTTTCGTC
GAACAGCATGAGACAGCGATCGCTCAGCTTACCACAATAGGCAAACCGCT
GGGTTCGCTCCATGAATGGGTTTAA
Amino acid sequence of the GFP expressed from the
ribosomal protein promoters
(SEQ ID NO: 17)
VNRNVLKNTGLKEIMSAKASVEGIVNNHVFSMEGFGKGNVLFGNQLMQIR
VTKGGPLPFAFDIVSIAFQYGNRTFTKYPDDIADYFVQSFPAGFFYERNL
RFEDGAIVDIRSDISLEDDKFHYKVEYRGNGFPSNGPVMQKAILGMEPSF
EVVYMNSGVLVGEVDLVYKLESGNYYSCHMKTFYRSKGGVKEFPEYHFIH
HRLEKTYVEEGSFVEQHETAIAQLTTIGKPLGSLHEWV
Nucleotide sequence of the FNA subtilisin protease
gene fused to the ribosomal protein promoters
(SEQ ID NO: 18)
ACAGAATAGTCTTTTAAGTAAGTCTACTCTGAATTTTTTTAAAAGGAGAG
GGTAAAGAGTGAGAAGCAAAAAATTGTGGATCAGTTTGCTGTTTGCTTTA
GCGTTAATCTTTACGATGGCGTTCGGCAGCACATCCTCTGCCCAGGCGGC
AGGGAAATCAAACGGGGAAAAGAAATATATTGTCGGGTTTAAACAGACAA
TGAGCACGATGAGCGCCGCTAAGAAGAAAGATGTCATTTCTGAAAAAGGC
GGGAAAGTGCAAAAGCAATTCAAATATGTAGACGCAGCTTCAGCTACATT
AAACGAAAAAGCTGTAAAAGAATTGAAAAAAGACCCGAGCGTCGCTTACG
TTGAAGAAGATCACGTAGCACATGCGTACGCGCAGTCCGTGCCTTACGGC
GTATCACAAATTAAAGCCCCTGCTCTGCACTCTCAAGGCTACACTGGATC
AAATGTTAAAGTAGCGGTTATCGACAGCGGTATCGATTCTTCTCATCCTG
ATTTAAAGGTAGCAGGCGGAGCCAGCATGGTTCCTTCTGAAACAAATCCT
TTCCAAGACAACAACTCTCACGGAACTCACGTTGCCGGCACAGTTGCGGC
TCTTAATAACTCAATCGGTGTATTAGGCGTTGCGCCAAGCGCATCACTTT
ACGCTGTAAAAGTTCTCGGTGCTGACGGTTCCGGCCAATACAGCTGGATC
ATTAACGGAATCGAGTGGGCGATCGCAAACAATATGGACGTTATTAACAT
GAGCCTCGGCGGACCTTCTGGTTCTGCTGCTTTAAAAGCGGCAGTTGATA
AAGCCGTTGCATCCGGCGTCGTAGTCGTTGCGGCAGCCGGTAACGAAGGC
ACTTCCGGCAGCTCAAGCACAGTGGGCTACCCTGGTAAATACCCTTCTGT
CATTGCAGTAGGCGCTGTTGACAGCAGCAACCAAAGAGCATCTTTCTCAA
GCGTAGGACCTGAGCTTGATGTCATGGCACCTGGCGTATCTATCCAAAGC
ACGCTTCCTGGAAACAAATACGGCGCGTTGAACGGTACATCAATGGCATC
TCCGCACGTTGCCGGAGCGGCTGCTTTGATTCTTTCTAAGCACCCGAACT
GGACAAACACTCAAGTCCGCAGCAGTTTAGAAAACACCACTACAAAACTT
GGTGATTCTTTCTACTATGGAAAAGGGCTGATCAACGTACAGGCGGCAGC
TCAGTAA
Amino acid sequence of the FNA subtilisin protease
expressed from the ribosomal protein promoters
(SEQ ID NO: 19)
VRSKKLWISLLFALALIFTMAFGSTSSAQAAGKSNGEKKYIVGFKQTMST
MSAAKKKDVISEKGGKVQKQFKYVDAASATLNEKAVKELKKDPSVAYVEE
DHVAHAYAQSVPYGVSQIKAPALHSQGYTGSNVKVAVIDSGIDSSHPDLK
VAGGASMVPSETNPFQDNNSHGTHVAGTVAALNNSIGVLGVAPSASLYAV
KVLGADGSGQYSWIINGIEWAIANNMDVINMSLGGPSGSAALKAAVDKAV
ASGVVVVAAAGNEGTSGSSSTVGYPGKYPSVIAVGAVDSSNQRASFSSVG
PELDVMAPGVSIQSTLPGNKYGALNGTSMASPHVAGAAALILSKHPNWTN
TQVRSSLENTTTKLGDSFYYGKGLINVQAAAQ
Nucleotide sequence of the ER11 subtilisin
protease gene fused to the ribosomal
protein promoters
(SEQ ID NO: 20)
GAATAGTCTTTTAAGTAAGTCTACTCTGAATTTTTTTAAAAGGAGAGGGT
AAAGAGTGAGAAGCAAAAAATTGTGGATCAGCTTGTTGTTTGCGTTAACG
TTAATCTTTACGATGGCGTTCAGCAACATGTCTGCGCAGGCTGCTGAAGA
AGCAAAAGAAAAATATTTAATTGGCTTTAATGAGCAGGAAGCTGTCAGTG
AGTTTGTAGAACAAGTAGAGGCAAATGACGGCGTCGCCATTCTCTCTGAG
GAAGAGGAAGTCGAAATTGAATTGCTTCATGAATTTGAAACGATTCCTGT
TTTATCCGTTGAGTTAAGCCCAGAAGATGTGGACGCGCTTGAACTCGATC
CAGCGATTTCTTATATTGAAGAGGATGCAGAAGTAACGACAATGGCGCAA
TCGGTACCATGGGGAATTAGCCGTGTGCAAGCCCCAGCTGCCCATAACCG
TGGATTGACAGGTTCTGGTGTAAAAGTTGCTGTCCTCGATACAGGTATTT
CCACTCATCCAGACTTAAATATTCGTGGTGGCGCTAGCTTTGTACCAGGG
GAACCATCCACTCAAGATGGGAATGGGCATGGCACGCATGTGGCTGGGAC
GATTGCTGCTTTAAACAATTCGATTGGCGTTCTTGGCGTAGCACCGAACG
CGGAACTATACGCTGTTAAAGTATTAGGGGCGAGCGGTATGGGTTCGGTC
AGCTCGATTGCCCAAGGATTGGAATGGGCAGGGAACAATGTTATGCACGT
TGCTAATTTGAGTTTAGGACTGCAGGCACCAAGTGCCACACTTGAGCAAG
CTGTTAATAGCGCGACTTCTAGAGGCGTTCTTGTTGTAGCGGCATCTGGC
AATTCAGGTGCAGGCTCAATCAGCTATCCGGCCCGTTATGCGAACGCAAT
GGCAGTCGGAGCTACTGACCAAAACAACAACCGCGCCAGCTTTTCACAGT
ATGGCGCAGGGCTTGACATTGTCGCACCAGGTGTAAACGTGCAGAGCACA
TACCCAGGTTCAACGTATGCCAGCTTAAACGGTACATCGATGGCTACTCC
TCATGTTGCAGGTGCAGCAGCCCTTGTTAAACAAAAGAACCCATCTTGGT
CCAATGTCCAAATCCGCAATCATCTAAAGAATACGGCAACGAGCTTAGGA
AGCACGAACTTGTATGGAAGCGGACTTGTCAATGCAGAAGCGGCAACACG
TTAA
Amino acid sequence of the ER11 subtilisin
protease expressed from the ribosomal
protein promoters
(SEQ ID NO: 21)
VRSKKLWISLLFALTLIFTMAFSNMSAQAAEEAKEKYLIGFNEQEAVSEF
VEQVEANDGVAILSEEEEVEIELLHEFETIPVLSVELSPEDVDALELDPA
ISYIEEDAEVTTMAQSVPWGISRVQAPAAHNRGLTGSGVKVAVLDTGIST
HPDLNIRGGASFVPGEPSTQDGNGHGTHVAGTIAALNNSIGVLGVAPNAE
LYAVKVLGASGMGSVSSIAQGLEWAGNNVMHVANLSLGLQAPSATLEQAV
NSATSRGVLVVAASGNSGAGSISYPARYANAMAVGATDQNNNRASFSQYG
AGLDIVAPGVNVQSTYPGSTYASLNGTSMATPHVAGAAALVKQKNPSWSN
VQIRNHLKNTATSLGSTNLYGSGLVNAEAATR

The different strains constructed from the fusion of the promoters to the target genes are listed in Table 2-2 below.

TABLE 2-2
List of strains
constructed

BG8000 PaprE-FNA
BG8000 PaprE-FNA
BG8010 PaprE-FNA
BG8010 PaprE-FNA
BG8000 PaprE-ER11
BG8000 PaprE-ER11
BG8010 PaprE-ER11
BG8010 PaprE-ER11
BG8010 PrpsD-FNA
BG8010 PrpsJ-FNA
BG8010 PrpsJ-GFP
BG8010 PrpsD-GFP
BG8010 PaprE-GFP

### Example 7

### Cell Density Measurements of GFP and FNA Expressing Strains

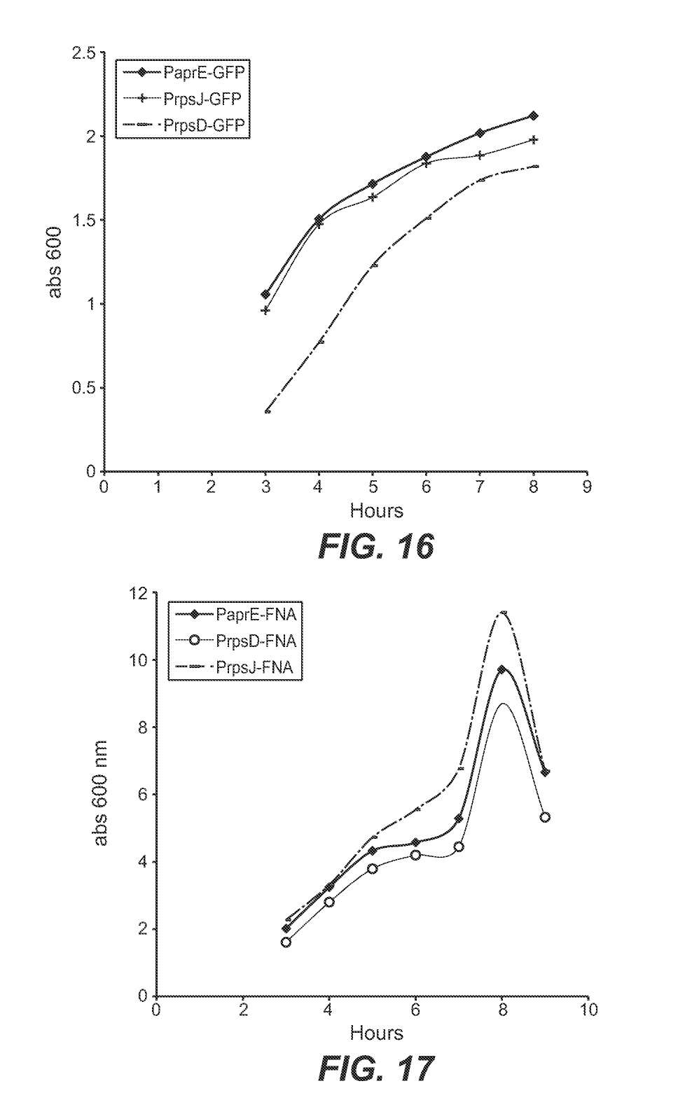

To test for cell growth, one colony each of the constructed strains was inoculated in Luria Broth containing 25 μg/mlchloramphenicol for strains expressing from aprE or rpoD promoters and grown overnight at 30° C. One ml of each pre-culture was used to inoculate 32 ml of 2×SNB medium (see composition below) and grown at 37° C. in shake flasks at 280 rpm, 70% humidity. At hourly intervals from 4 hours to 8 hours of growth, optical densities of each culture was measured at 600 nm using a SpectraMax reader. The cell density measurements of GFP and FNA expressing strains are shown in FIGS. 16 (GFP) and 17 (FNA). The growth of strains containing the different constructs was comparable.

2×SNB Medium:

Stock solutions (filter sterilized): 25×SNB salts-CaCl₂*2H₂O (3.7 g/L), FeSO₄*7H₂O (9.6 mg/L), MnCl₂*4H₂O (6 mg/L), KCl (25 g/L), MgSO₄*7H₂O (3.26 g/L), Maltrin 150 10%. Prepare 500 mL of 16 g/L solution of Difco Nutrient Broth, autoclave, add 20 mL 25×SNB salts, and 25 mL 10% Maltrin 150.

### Example 8

### Protein Expression of GFP and FNA from Ribosomal Promoters

The production of FNA or intracellular expression of GFP driven by the selected promoters was tested in BG8000 and BG8010 strains. The cells were grown as described for the cell density measurements in Example 7. At hourly intervals from 4 hours to 8 hours of growth, supernatants of cultures were analyzed for AAPF activity (subtilisin expression). GFP expression was measured as Relative Fluorescence Units (RFU) expressed in the cell.

The AAPF activity of a sample was measured as the rate of hydrolysis of N-succinyl-L-alanyl-L-alanyl-L-prolyl-L-phenyl-p-nitroanilide (suc-AAPF-pNA). The reagent solutions used were: 100 mM Tris/HCl, pH 8.6, containing 0.005% TWEEN®-80 (Tris dilution buffer and 160 mM suc-AAPF-pNA in DMSO (suc-AAPF-pNA stock solution) (Sigma: S-7388). To prepare a suc-AAPF-pNA working solution, 1 ml suc-AAPF-pNA stock solution was added to 100 ml Tris/HCl buffer and mixed well for at least 10 seconds. The assay was performed by diluting the samples in the assay buffer (5 μl in 195 μl). Then, 180 μl of assay buffer with AAPF substrate was added to 20 μl of the diluted sample arrayed in a microtiter plate. The solutions were mixed for 5 sec., and the absorbance change in kinetic mode (20 readings in 5 minutes) was read at 405 nm in a SpectraMax reader, at 25° C.

For measuring GFP expression in RFU, 150 μl of each culture sample was loaded into a microtiter plate and fluorescence measurements were taken using the SpectraMax reader using an excitation wavelength at 485 nm, emission wavelength at 508 nm, with a cutoff at 495 nm.

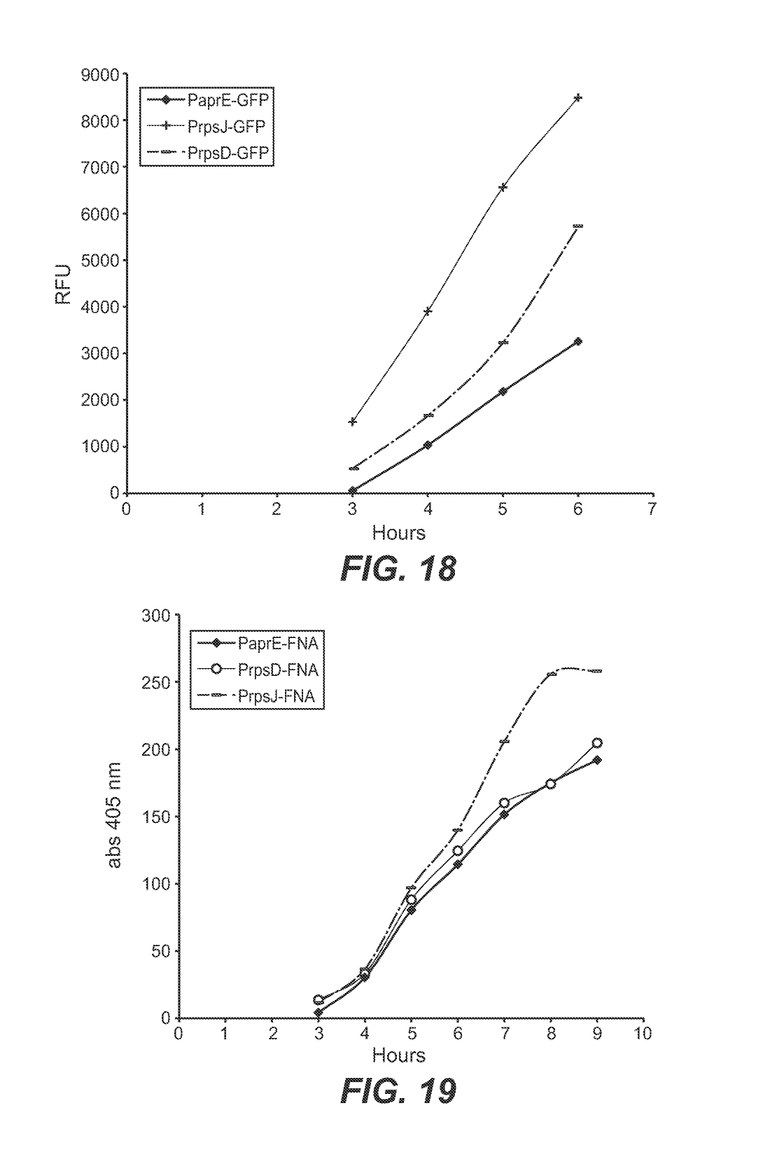

Expression of GFP and FNA from the different promoters is shown in FIGS. 18 (GFP) and 19 (FNA). Protein expression from non-amplified ribosomal protein promoter was higher than that seen from amplified aprE promoter.

### Example 9

### Protein Expression from SigmaA Dependent Promoter

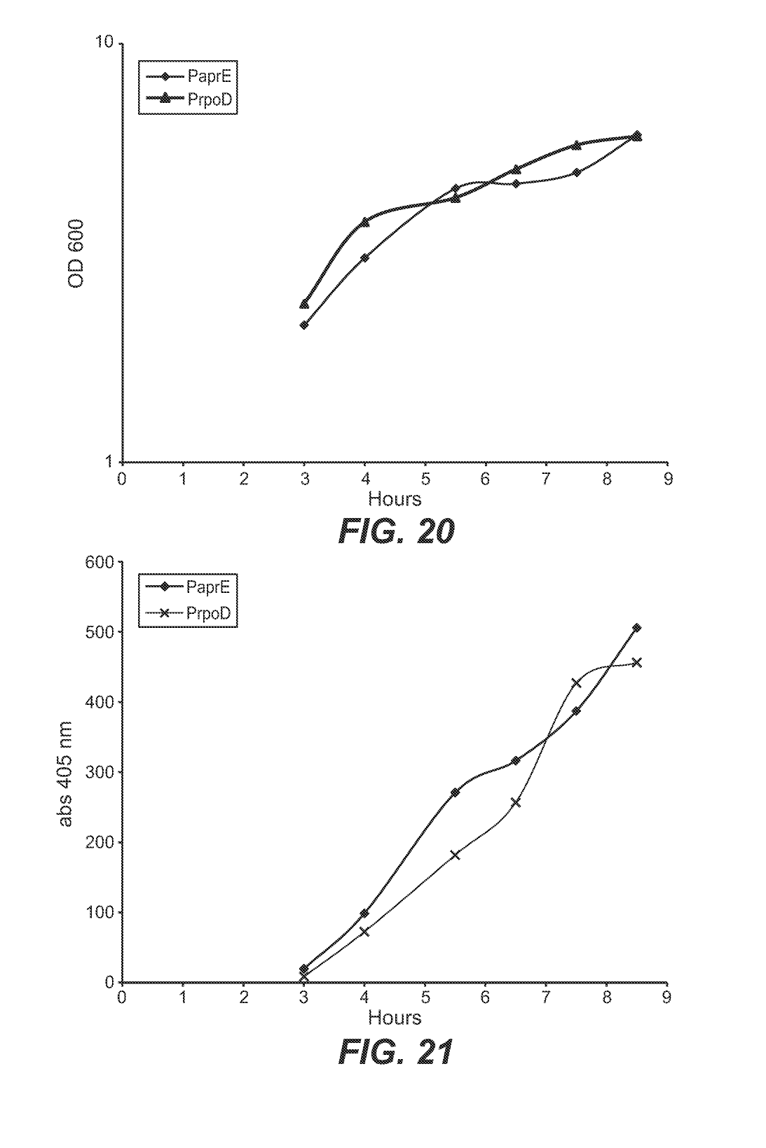

As different levels of protein expression are observed from different promoters, this experiment compared FNA expression from amplified rpoD promoter (a promoter for the sigmaA housekeeping sigma factor in B subtilis) with that from amplified aprE promoter. BG8010 strains expressing FNA from rpoD and aprE were amplified using 25 μg/mL chloramphenicol. Cell density measurements and FNA expression was studied as described in Examples 7 and 8 respectively. Results are shown in FIGS. 20 and 21. Cell growth from all strains was comparable.

1. An isolated nucleic acid comprising a B. subtilis ribosomal promoter operably linked to a nucleic acid encoding a heterologous protein of interest.

2. The isolated nucleic acid of claim 1, wherein said nucleic acid is a ribosomal RNA promoter or a ribosomal protein promoter.

3. The isolated nucleic acid of claim 1, wherein said nucleic acid is a rrn ribosomal RNA promoter.

4. The isolated nucleic acid of claim 1, wherein said nucleic acid is a rrnB, rrnI, or rrnE ribosomal RNA promoter.

5. The isolated nucleic acid of claim 1, wherein said nucleic acid is a rrnI ribosomal RNA promoter.

6. The isolated nucleic acid of claim 1, wherein said nucleic acid is a rpsD or rpsJ ribosomal protein promoter.

7. The isolated nucleic acid of claim 1, wherein the nucleic acid comprises the nucleotide sequence of any one of SEQ ID NOs: 1-6, a subsequence of any one of SEQ ID NOs: 1-6 that retains promoter activity, a nucleic acid that is at least 60% homologous to any one of SEQ ID NOs: 1-6, or a nucleic acid that hybridizes under medium stringency conditions with any one of SEQ ID NOs: 1-6 or the subsequence thereof that retains promoter activity, or combinations thereof of any of the above.

8. The isolated nucleic acid of claim 0, wherein the nucleic acid comprises the nucleotide sequence of SEQ ID NO: 3 or a subsequence thereof retaining promoter activity.

9. The isolated nucleic acid of claim 1, wherein the nucleic acid comprises the nucleotide sequence of any one of SEQ ID NOs: 13-14, a subsequence of any one of SEQ ID NOs: 13-14 that retains promoter activity, a nucleic acid that is at least 60% homologous to any one of SEQ ID NOs: 13-14, or a nucleic acid that hybridizes under medium stringency conditions with any one of SEQ ID NOs: 13-14 or the subsequence thereof that retains promoter activity, or combinations thereof of any of the above.

10. The isolated nucleic acid of claim 1, wherein the nucleic acid comprises a nucleic acid that is at least 60%, 70%, 80%, 90%, 93%, 95%, 97%, or 99% homologous to any one of SEQ ID NOs: 13-14.

11. The isolated nucleic acid of any of the preceding claims, wherein the protein of interest is selected from the group consisting of a hormone, enzyme, growth factor, reporter gene, and cytokine.

12. The isolated nucleic acid of any of the preceding claims, wherein the protein of interest is an enzyme.

13. The isolated nucleic acid of any of the preceding claims, wherein the protein of interest is selected from the group consisting of a protease, cellulase, amylase, xylanase, phytase, mannanase, hemicellulase, carbohydrase, hydrolase, esterase, oxidase, permease, pullulanase, laccase, lipase, reductase, isomerase, epimerase, tautomerase, transferase, kinase, and phosphatase.

14. The isolated nucleic acid of any of the preceding claims, wherein the protein of interest is a protease.

15. The isolated nucleic acid of any of the preceding claims, wherein the protease is subtilisin.

16. The isolated nucleic acid of any of the preceding claims, wherein the protease is encoded by SEQ ID NO: 9, 11, 18, or 20.

17-92. (canceled)


SEQUENCE LISTING (37 sequences)

SEQ ID NO: 1 - DNA, 65 bp, Artificial Sequence
  Synthetic oligonucleotide P1 rrnB
atagattttttttaaaaaactattgcaataaataaatacaggtgttatattattaaacgtcgctg

SEQ ID NO: 2 - DNA, 66 bp, Artificial Sequence
  Synthetic oligonucleotide P1 rrnI
cacatacagcctaaattgggtgttgaccttttgataatatccgtgatatattattattcgtcgctg

SEQ ID NO: 3 - DNA, 64 bp, Artificial Sequence
  Synthetic oligonucleotide P2 rrnI
ttaaatactttgaaaaaagttgttgacttaaaagaagctaaatgttatagtaataaagctgctt

SEQ ID NO: 4 - DNA, 65 bp, Artificial Sequence
  Synthetic oligonucleotide P1 rrnE
ataaaaaaatacaggaaaagtgttgaccaaataaaacaggcatggtatattattaaacgtcgctg

SEQ ID NO: 5 - DNA, 65 bp, Artificial Sequence
  Synthetic oligonucleotide P2 rrnE
aacaaaaaagttttcctaaggtgtttacaagattttaaaaatgtgtataataagaaaagtcgaat

SEQ ID NO: 6 - DNA, 64 bp, Artificial Sequence
  Synthetic oligonucleotide P3 rrnE
tcgaaaaaacattaaaaaacttcttgactcaacatcaaatgatagtatgatagttaagtcgctc

SEQ ID NO: 7 - DNA, 775 bp, Artificial Sequence
  Synthetic construct Nucleotide sequence of the GFP gene fused to the ribosomal RNA and protein promoters
acagaatagtcttttaagtaagtctactctgaatttttttaaaaggagagggtaaagagtgaatagaaatgttcttaaaaatactggtctgaaggagatcatgtcagcgaaagcgtctgtggaaggtattgtgaacaatcacgtattctcaatggaggggtttggaaagggaaatgttttgtttggtaaccagttaatgcaaattcgagttaccaaaggcggcccacttccatttgccttcgacatcgtaagcatcgccttccagtacggcaatcgcacctttacgaaatatcctgatgatatcgccgactatttcgtgcaatcgtttccagcgggctttttctatgaaagaaatctgcggtttgaagatggcgcaatcgttgatatacgttcagacatcagtctggaggatgacaagtttcactataaagtggagtatcgaggaaacggatttccgtctaacgggcctgtcatgcaaaaagctattttgggcatggagccgtcttttgaagtggtttatatgaatagcggcgtccttgtaggggaagtggatttagtttataagctggaaagcggaaattattattcatgccatatgaaaaccttctatagatcaaagggcggagtgaaagaatttccagaatatcactttattcatcatagactggagaaaacgtatgttgaagaaggttctttcgtcgaacagcatgagacagcgatcgctcagcttaccacaataggcaaaccgctgggttcgctccatgaatgggtttaa

SEQ ID NO: 8 - protein, 238 aa, Artificial Sequence
  Synthetic construct Amino acid sequence of the GFP expressed from the ribosomal RNA and protein promoters
VNRNVLKNTGLKEIMSAKASVEGIVNNHVFSMEGFGKGNVLFGNQLMQIRVTKGGPLPFAFDIVSIAFQYGNRTFTKYPDDIADYFVQSFPAGFFYERNLRFEDGAIVDIRSDISLEDDKFHYKVEYRGNGFPSNGPVMQKAILGMEPSFEVVYMNSGVLVGEVDLVYKLESGNYYSCHMKTFYRSKGGVKEFPEYHFIHHRLEKTYVEEGSFVEQHETAIAQLTTIGKPLGSLHEWV

SEQ ID NO: 9 - DNA, 1207 bp, Artificial Sequence
  Synthetic construct Nucleotide sequence of the FNA subtilisin protease gene fused to the ribosomal RNA and protein promoters
acagaatagtcttttaagtaagtctactctgaatttttttaaaaggagagggtaaagagtgagaagcaaaaaattgtggatcagtttgctgtttgctttagcgttaatctttacgatggcgttcggcagcacatcctctgcccaggcggcagggaaatcaaacggggaaaagaaatatattgtcgggtttaaacagacaatgagcacgatgagcgccgctaagaagaaagatgtcatttctgaaaaaggcgggaaagtgcaaaagcaattcaaatatgtagacgcagcttcagctacattaaacgaaaaagctgtaaaagaattgaaaaaagacccgagcgtcgcttacgttgaagaagatcacgtagcacatgcgtacgcgcagtccgtgccttacggcgtatcacaaattaaagcccctgctctgcactctcaaggctacactggatcaaatgttaaagtagcggttatcgacagcggtatcgattcttctcatcctgatttaaaggtagcaggcggagccagcatggttccttctgaaacaaatcctttccaagacaacaactctcacggaactcacgttgccggcacagttgcggctcttaataactcaatcggtgtattaggcgttgcgccaagcgcatcactttacgctgtaaaagttctcggtgctgacggttccggccaatacagctggatcattaacggaatcgagtgggcgatcgcaaacaatatggacgttattaacatgagcctcggcggaccttctggttctgctgctttaaaagcggcagttgataaagccgttgcatccggcgtcgtagtcgttgcggcagccggtaacgaaggcacttccggcagctcaagcacagtgggctaccctggtaaatacccttctgtcattgcagtaggcgctgttgacagcagcaaccaaagagcatctttctcaagcgtaggacctgagcttgatgtcatggcacctggcgtatctatccaaagcacgcttcctggaaacaaatacggcgcgttgaacggtacatcaatggcatctccgcacgttgccggagcggctgctttgattctttctaagcacccgaactggacaaacactcaagtccgcagcagtttagaaaacaccactacaaaacttggtgattctttctactatggaaaagggctgatcaacgtacaggcggcagctcagtaa

SEQ ID NO: 10 - protein, 382 aa, Artificial Sequence
  Synthetic construct Amino acid sequence of the FNA subtilisin protease expressed from the ribosomal RNA and protein promoters
VRSKKLWISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAYAQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 11 - DNA, 1204 bp, Artificial Sequence
  Synthetic construct Nucleotide sequence of the ER11 subtilisin protease gene fused to the ribosomal RNA and protein promoters
gaatagtcttttaagtaagtctactctgaatttttttaaaaggagagggtaaagagtgagaagcaaaaaattgtggatcagcttgttgtttgcgttaacgttaatctttacgatggcgttcagcaacatgtctgcgcaggctgctgaagaagcaaaagaaaaatatttaattggctttaatgagcaggaagctgtcagtgagtttgtagaacaagtagaggcaaatgacggcgtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattagccgtgtgcaagccccagctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggctgggacgattgctgctttaaacaattcgattggcgttcttggcgtagcaccgaacgcggaactatacgctgttaaagtattaggggcgagcggtatgggttcggtcagctcgattgcccaaggattggaatgggcagggaacaatgttatgcacgttgctaatttgagtttaggactgcaggcaccaagtgccacacttgagcaagctgttaatagcgcgacttctagaggcgttcttgttgtagcggcatctggcaattcaggtgcaggctcaatcagctatccggcccgttatgcgaacgcaatggcagtcggagctactgaccaaaacaacaaccgcgccagcttttcacagtatggcgcagggcttgacattgtcgcaccaggtgtaaacgtgcagagcacatacccaggttcaacgtatgccagcttaaacggtacatcgatggctactcctcatgttgcaggtgcagcagcccttgttaaacaaaagaacccatcttggtccaatgtccaaatccgcaatcatctaaagaatacggcaacgagcttaggaagcacgaacttgtatggaagcggacttgtcaatgcagaagcggcaacacgttaa

SEQ ID NO: 12 - protein, 382 aa, Artificial Sequence
  Synthetic construct Amino acid sequence of the ER11 subtilisin protease expressed from the ribosomal RNA and protein promoters
VRSKKLWISLLFALTLIFTMAFSNMSAQAAEEAKEKYLIGFNEQEAVSEFVEQVEANDGVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGMGSVSSIAQGLEWAGNNVMHVANLSLGLQAPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 13 - DNA, 112 bp, Artificial Sequence
  Synthetic oligonucleotide rpsD
gtttttatcacctaaaagtttaccactaatttttgtttattatatcataaacggtgaagcaataatggaggaatggttgacttcaaaacaaataaattatataatgaccttt

SEQ ID NO: 14 - DNA, 303 bp, Artificial Sequence
  Synthetic oligonucleotide rpsJ
gtaccgtgtgttttcatttcagggaaacatgacttaattgttcctgcagaaatatcgaaacagtattatcaagaacttgaggcacctgaaaagcgctggtttcaatttgagaattcagctcacaccccgcatattgaggagccatcattattcgcgaacacattaagtcggcatgcacgcaaccatttatgatagatccttgataaataagaaaaacccctgtataataaaaaaagtgtgcaaatgatgcatattttaaataagtcttgcaacatgcgcctattttctgtataatggtgtata

SEQ ID NO: 15 - DNA, 103 bp, Artificial Sequence
  Synthetic oligonucleotide rpoD (P1)
aacatataactcaggacgctctatcctgggtttttggctgtgccaaaagggaataatgaaaaacaatagcatctttgtgaagtttgtattataataaaaaatt

SEQ ID NO: 16 - DNA, 775 bp, Artificial Sequence; identical to SEQ ID NO: 7
  Synthetic construct Nucleotide sequence of the GFP gene fused to the ribosomal protein promoters

SEQ ID NO: 17 - protein, 238 aa, Artificial Sequence; identical to SEQ ID NO: 8
  Synthetic construct Amino acid sequence of the GFP expressed from the ribosomal protein promoters

SEQ ID NO: 18 - DNA, 1207 bp, Artificial Sequence; identical to SEQ ID NO: 9
  Synthetic construct Nucleotide sequence of the FNA subtilisin protease gene fused to the ribosomal protein promoters

SEQ ID NO: 19 - protein, 382 aa, Artificial Sequence; identical to SEQ ID NO: 10
  Synthetic construct Amino acid sequence of the FNA subtilisin protease expressed from the ribosomal protein promoters

SEQ ID NO: 20 - DNA, 1204 bp, Artificial Sequence; identical to SEQ ID NO: 11
  Synthetic construct Nucleotide sequence of the ER11 subtilisin protease gene fused to the ribosomal protein promoters

SEQ ID NO: 21 - protein, 382 aa, Artificial Sequence; identical to SEQ ID NO: 12
  Synthetic construct Amino acid sequence of the ER11 subtilisin protease expressed from the ribosomal protein promoters

SEQ ID NO: 22 - DNA, 20 bp, Artificial Sequence
  Synthetic oligonucleotide
aaawwtwttttnnnaaannn

SEQ ID NO: 23 - DNA, 65 bp, Artificial Sequence
  Synthetic oligonucleotide A-1
atcatttaattgatattatgtattgacttagacaactgaaggtgttattctaatatacgtcgctg

SEQ ID NO: 24 - DNA, 64 bp, Artificial Sequence
  Synthetic oligonucleotide D-1
ggatattcttttaaaaaaggtgttgactctgattcttgaccgtgttatattattaacgtcctga

SEQ ID NO: 25 - DNA, 64 bp, Artificial Sequence
  Synthetic oligonucleotide J-1
tagtatttcttcaaaaaaactattgcactattatttactaggtggtatattattattgttgccg

SEQ ID NO: 26 - DNA, 64 bp, Artificial Sequence
  Synthetic oligonucleotide O-1
gcgcttttttgtgtcataaccctttacagtcataaaaattatggtataatcatttctgttgtct

SEQ ID NO: 27 - DNA, 51 bp, Artificial Sequence
  Synthetic consensus sequence
atttttaaaaaagtttgacaattaaaaagtgtatattattatacgtcgctg

SEQ ID NO: 28 - DNA, 64 bp, Artificial Sequence
  Synthetic oligonucleotide A-2
aaaagaaaatgctaaaaagttgttgacagtagcggcggtaaatgttatgataataaagtcgctt

SEQ ID NO: 29 - DNA, 64 bp, Artificial Sequence
  Synthetic oligonucleotide B-2
caaaacaacttgaaaaaagttgttgacaaaaaagaagctgaatgttatattagtaaagctgctt

SEQ ID NO: 30 - DNA, 64 bp, Artificial Sequence; identical to SEQ ID NO: 3
  Synthetic oligonucleotide I-2

SEQ ID NO: 31 - DNA, 66 bp, Artificial Sequence
  Synthetic oligonucleotide W-2
ccaaaagtttttaaaaaagttgttgactttgaagaagtgacgtttatactaataaagttgctttaa

SEQ ID NO: 32 - DNA, 67 bp, Artificial Sequence
  Synthetic oligonucleotide H-2
caaaagtttttaaaaaaggttattgactttgaagaagtgacattgtatactaataaagttgctttaa

SEQ ID NO: 33 - DNA, 67 bp, Artificial Sequence
  Synthetic oligonucleotide G-2
gtgtaattttttaaaaaagttattgactttgaagaagtgacattgtatactaataaagttgctttaa

SEQ ID NO: 34 - DNA, 64 bp, Artificial Sequence
  Synthetic oligonucleotide D-2
ggaaaataaatcaaaaaaacatttgacaaaagaaagtcaaaatgttatattaataaagtcgcgt

SEQ ID NO: 35 - DNA, 64 bp, Artificial Sequence
  Synthetic oligonucleotide J-2
aaaagaacttcaaaaaaagttattgacttcactgagtcaaggagttataataataaagacgtac

SEQ ID NO: 36 - DNA, 64 bp, Artificial Sequence
  Synthetic oligonucleotide O-2
taaaaactttttcaaaaaagtattgacctagttaactaaaaatgttactattaagtagtcgctt

SEQ ID NO: 37 - DNA, 54 bp, Artificial Sequence
  Synthetic consensus sequence
aaaaaatttaaaaaagttttgactaagaaaaatgttataataataaagtcgctt